(19) United States
(12) Patent Application Publication
(10) Pub. No.: US 2026/0285822 A1
(43) Pub. Date: Sep. 24, 2026

(54) START-UP METHOD FOR A PROCESS FOR PREPARING AN OLEFIN OXIDE

(71) Applicants: BASE SE, Ludwigshafen am Rhein (DE); Dow Global Technologies LLC, Midland, MI (US)

(72) Inventors: Joaquim Henrique TELES, Ludwigshafen am Rhein (DE); Jaroslaw Michael MORMUL, Ludwigshafen am Rhein (DE); Massimo BERGAMO, Stade (DE); Meinolf WEIDENBACH, Stade (DE); Dylan SEGERS, Antwerpen (BE); Franciscus Johannes Robertus VAN NEER, Hoek (NL); Trees Maria DE BAERDEMAEKER, Ludwigshafen am Rhein (DE); Andrei-Nicolae PARVULESCU, Ludwigshafen am Rhein (DE)

(21) Appl. No.: 19/472,417

(22) PCT Filed: Apr. 5, 2024
(86) PCT No.: PCT/EP2024/059329

§ 371 Date: Oct. 3, 2025

(30) Foreign Application Priority Data
Apr. 6, 2023 (EP) 23166987.0

(51) Int. Cl. C07D 301/12; C07D 301/36

(52) U.S. Cl. CPC C07D 301/12; C07D 301/36

(57) ABSTRACT

In a first aspect, the invention relates to a start-up method for a process for preparing an olefin oxide comprising a normal run stage, wherein the normal run stage comprises providing olefin, hydrogen peroxide, additive, water and organic solvent into an epoxidation zone comprising an heterogeneous epoxidation catalyst, so that a reaction mixture comprising olefin, hydrogen peroxide, additive, water and organic solvent is formed and subjecting the reaction mixture to epoxidation reaction conditions in the epoxidation zone, thereby obtaining a mixture comprising olefin oxide and organic solvent; wherein the start-up method comprises (a) providing an organic solvent, an additive, water and optionally an olefin for a first period of time P1 to the epoxidation zone, so that a first mixture 1 is formed, which comprises organic solvent, additive, water and optionally olefin and contacting the first mixture 1 over P1 under epoxidation reaction conditions in the epoxidation zone with the heterogeneous epoxidation catalyst; wherein the additive releases or forms one or more cation(s) M, wherein at the end of P1 the heterogeneous epoxidation catalyst has been contacted with a molar amount M per weight catalyst in the range of from 2500 to 4500 μmol M/kg catalyst. A second aspect of the invention is directed to a process for preparing an olefin oxide comprising a normal run stage and a start-up stage, wherein the normal run stage comprises (A) providing olefin, hydrogen peroxide, additive water and organic solvent into an epoxidation zone comprising an heterogeneous epoxidation catalyst, so that a reaction mixture comprising olefin, hydrogen peroxide, additive, water and organic solvent is formed; (B) subjecting the reaction mixture from (A) to epoxidation reaction conditions in the epoxidation zone, thereby obtaining a mixture comprising olefin oxide, additive, water and organic solvent; (C) removing an effluent stream from the epoxidation zone, comprising olefin oxide, additive, water and organic solvent; wherein the start-up stage comprises a step (a). A third aspect of the invention relates to an olefin oxide obtained or obtainable from the process of the second aspect.

In a first aspect, the invention relates to a start-up method for a process for preparing an olefin oxide comprising a normal run stage, wherein the normal run stage comprises providing olefin, hydrogen peroxide, additive, water and organic solvent into an epoxidation zone comprising an heterogeneous epoxidation catalyst, so that a reaction mixture comprising olefin, hydrogen peroxide, additive, water and organic solvent is formed and subjecting the reaction mixture to epoxidation reaction conditions in the epoxidation zone, thereby obtaining a mixture comprising olefin oxide and organic solvent; wherein the start-up method comprises (a) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst; wherein the additive releases or forms one or more cation(s) M, wherein at the end of P₁ the heterogeneous epoxidation catalyst has been contacted with a molar amount M per weight catalyst in the range of from 2500 to 4500 μmol M/kg catalyst. A second aspect of the invention is directed to a process for preparing an olefin oxide comprising a normal run stage and a start-up stage, wherein the normal run stage comprises (A) providing olefin, hydrogen peroxide, additive water and organic solvent into an epoxidation zone comprising an heterogeneous epoxidation catalyst, so that a reaction mixture comprising olefin, hydrogen peroxide, additive, water and organic solvent is formed; (B) subjecting the reaction mixture from (A) to epoxidation reaction conditions in the epoxidation zone, thereby obtaining a mixture comprising olefin oxide, additive, water and organic solvent; (C) removing an effluent stream from the epoxidation zone, comprising olefin oxide, additive, water and organic solvent; wherein the start-up stage comprises a step (a). A third aspect of the invention relates to an olefin oxide obtained or obtainable from the process of the second aspect.

Olefin oxides such as propylene oxide are important intermediates in the chemical industry. A suitable process for the preparation of olefin oxide starts from the respective olefin and makes use of hydrogen peroxide as oxidizing agent, organic solvents, water and heterogeneous epoxidation catalysts such as titanium containing zeolites. Due to the importance for industrial-scale processes, it is desired to carry out such epoxidation reactions as efficiently as possible.

Any process and even more important each industrial scale process comprises at least three stages, that is a start-up stage, wherein the reaction is started, a normal run stage, wherein the reaction is carried out so that the desired product is obtained, and finally a shutdown stage, where the reaction is terminated and the reaction vessel may be emptied, for example, in order to enable catalyst regeneration, replacement etc.

The start-up stage, since it defines the conditions under which the catalyst in the reactor has to operate, is of major importance. Especially when, as it is often the case, epoxidations are carried out in several reactors in parallel, the start-up is relevant, since normally, any epoxidation process is part of a highly integrated system, wherein reactors are sequentially started and shutdown. In case one of a plurality of reactors has to be started, while one or more of the others are in operation, it is mandatory that this reactor do not negatively impair the overall balance.

EP 0 230 949 81 discloses a process for the epoxidation of olefinic compounds comprising reacting in a reaction zone one or more olefinic compounds with hydrogen peroxide introduced as such or produced by substances which can produce it at the reaction conditions, in presence of a synthetic zeolite as catalyst, characterized in that the catalyst is neutralized, as to its acidity, with neutralizing agents before and/or during the reaction. As suitable neutralizing agents, inter alia, strong or weak bases are described. However, regarding a neutralization during and before reactions, it is only disclosed that for a batchwise epoxidation reaction, a pretreatment of the catalyst by using a slurry of the catalyst in a diluted solution of the neutralizing agent, followed by repeated rinsing of the catalyst with water is required, whereas for a continuous epoxidation reaction, it is indicated that addition of the neutralizing agent only during reaction together with the hydrogen peroxide is sufficient.

The technical problem underlying the present invention was thus to provide an improved method for starting an epoxidation reaction.

Accordingly, in a first aspect, the invention is directed to a start-up method for a process for preparing an olefin oxide comprising a normal run stage, wherein the normal run stage comprises providing olefin, hydrogen peroxide, additive, water and organic solvent into an epoxidation zone comprising an heterogeneous epoxidation catalyst, so that a reaction mixture comprising olefin, hydrogen peroxide, additive, water and organic solvent is formed and subjecting the reaction mixture to epoxidation reaction conditions in the epoxidation zone, thereby obtaining a mixture comprising olefin oxide and organic solvent;
wherein the start-up method comprises
(a) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst;
wherein the additive releases or forms one or more cation(s) M, wherein at the end of P₁ the heterogeneous epoxidation catalyst has been contacted with a molar amount M per weight catalyst in the range of from 2500 to 4500 μmol M/kg catalyst.

The first mixture 1 is essentially free of hydrogen peroxide. “Essentially free of hydrogen peroxide” regarding means that said first mixture 1 comprises less than 0.2 weight-%, preferably less than 0.1 weight-%, more preferably less than 0.05 weight-%, of hydrogen peroxide, based on the total weight of the first mixture 1.

It was surprisingly found that using a start-up procedure with start of additive feed prior to start of hydrogen peroxide feed helped significantly to minimize the propylene oxide losses. Especially, it proved effective to contact the heterogeneous epoxidation catalyst prior to start of hydrogen peroxide feed with a molar amount M, especially K⁺, per weight catalyst in the range of from 2500 to 4500 μmol M/kg catalyst, in view of avoidance of propylene oxide losses (selectivity to propylene oxide) and regarding reduction of by-products.

In some preferred embodiments of the start-up method, the additive is selected from the group consisting of potassium salt of an inorganic acid, potassium salt of an organic acid, ammonia, ammonium salt of an inorganic acid, ammonium salt of an organic acid and mixtures of two or more thereof;
wherein the additive is preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof,
more preferably form the group consisting of potassium dihydrogen phosphate, dipotassium hydrogen phosphate, dipotassium etidronate, ammonia and mixtures of two or more thereof;
wherein the additive more preferably comprises at least dipotassium etidronate; wherein more preferably in the range of from 95 to 100 weight-% of the additive are dipotassium etidronate.

In some preferred embodiments of the start-up method, the additive is in case of ammonia provided in pure form or in aqueous solution, wherein the additive is in case of being selected from the group consisting of potassium salt of an inorganic acid, potassium salt of an organic acid, ammonium salt of an inorganic acid, ammonium salt of an organic acid or any mixture comprising two or more salts of these acids or any mixture comprising ammonia and at least one salts of these acids provided in aqueous solution; wherein M represents in case of a potassium salt K⁺, or, in case of ammonia or ammonium salts, M represents the sum of NH₄⁺ and NH₃.

Etidronic acid is a tetrabasic acid with pKs values of 1.35±0.08; 2.87; 7.03±0.01; 11.3. Each of the above-mentioned ammonia, potassium salts and ammonium salts, when in aqueous solution, dissociates to release or forms at least to some extend anion(s) and one or more cation(s). For example, ammonia forms to some extent NH₄⁺, ammonium salts dissociate to some extent to NH₄⁺ and one or more anion(s), or potassium salts dissociate to some extent to release K⁺ and one or more anion(s), the positive charge compensated by the appropriate equivalent of anion(s)). Regarding ammonia and ammonium salts, due to the dissociation equilibrium between NH₃ and NH₄⁺, the sum of NH₃ and NH₄⁺ has to be considered for M.

In some preferred embodiments of the start-up method, the additive is selected from the group consisting of potassium salt of an inorganic acid, potassium salt of an organic acid, ammonium salt of an inorganic acid, ammonium salt of an organic acid, a mixture comprising two or more of these acid salts and a mixture comprising ammonia and at least one of these acid salts, preferably from the group consisting of potassium salt of an inorganic acid, potassium salt of an organic acid, ammonium salt of an inorganic acid, ammonium salt of an organic acid, and a mixture comprising two or more of these acid salts, and is provided as an aqueous solution, wherein the aqueous solution of additive preferably comprises in the range of from 0.01 to 5 weight-%, more preferably in the range of from 0.5 to 3 weight-%, more preferably in the range of from 1 to 2 weight-%, of additive, based on the overall weight of the aqueous solution.

In some preferred embodiments of the start-up method, the additive is ammonia, which is provided in pure form or in aqueous solution, wherein the aqueous solution of ammonia preferably comprises in the range of from 0.01 to 32 weight-% of ammonia, based on the overall weight of the aqueous solution.

“Pure form” regarding ammonia means that a composition comprising ammonia is used, which comprises in the range of from 95 to 100 weight-%, preferably in the range of from 96 to 100 weight-%, more preferably in the range of from 97 to 100 weight-%, more preferably in the range of from 98 to 100 weight-%, more preferably in the range of from 99 to 100 weight-%, more preferably in the range of from 99.5 to 100 weight-%, of ammonia, based on the overall weight of the composition being 100 weight-%, the remainder up to 100 weight-% preferably being water.

In some preferred embodiments of the start-up method, step (a) comprises
(a.1) providing organic solvent, an additive and water for a period of time P_{1.1} to the epoxidation zone, so that a mixture 1.1 is formed, which comprises organic solvent, additive and water and contacting the mixture 1.1 over P_{1.1} under epoxidation reaction conditions in the epoxidation zone with the heterogeneous epoxidation catalyst;
(a.2) providing organic solvent, additive, water and olefin for a second period of time P_{1.2} to the epoxidation zone, so that a mixture 1.2 is formed, which comprises organic solvent, additive, water and olefin, and contacting the second mixture 1.2 over P_{1.2} under epoxidation reaction conditions in the epoxidation zone with the heterogeneous epoxidation catalyst.

Preferably, the mixture 1.1 formed in (a. 1) is essentially free of olefin and essential free of hydrogen peroxide. The mixture 1.2 formed in (a.2) is essentially free of hydrogen peroxide “Essentially free of hydrogen peroxide” regarding the mixture 1.1 in (a.1) and the mixture 1.2 in (a.2) means that said mixture comprises less than 0.2 weight-%, preferably less than 0.1 weight-%, more preferably less than 0.05 weight-%, of hydrogen peroxide, based on the total weight of the mixture. “Essentially free of olefin” regarding the mixture 1.1 in (a. 1) means that said mixture comprises less than 0.2 weight-%, preferably less than 0.1 weight-%, more preferably less than 0.05 weight-%, of olefin, based on the total weight of the mixture.

In some preferred embodiments of the start-up method, the additive is provided in at least one of (a), (a.1), (a.2), preferably in (a) or in (a.1), (a.2) respectively, in a molar ratio M:hydrogen peroxide intended to be provided in the normal run stage in the range of from 1×10⁻⁵:1 to 1×10⁻³:1.

A molar ratio of M:hydrogen peroxide in the range of from 1.5×10⁻⁵:1 to 4.5×10⁻³:1 showed to be most favorable results regarding propylene oxide selectivity as well as regarding reduction of by-products.

In the normal run stage of the process for preparing an olefin oxide, the additive is preferably used so that a specific molar ratio of M to the hydrogen peroxide is established, which is preferably a molar ratio M:hydrogen peroxide in the normal run stage in the range of from 1×10⁻⁵:1 to 1×10⁻³:1. The expression “the additive is provided in at least one of (a), (a.1), (a.2), preferably in (a) or in (a.1), (a.2) respectively, in a molar ratio M:hydrogen peroxide intended to be provided in the normal run stage” means for the start-up method, that per mol of hydrogen peroxide intended to be used in the normal run stage, the respective molar amount of M is already used in the start-up stage. If, for example, it is intended for the normal run stage to use an aqueous H₂O₂ solution (40 weight-% H₂O₂) fed with a flow rate of 94 g/h in the normal run stage, i.e. 1.106 mol H₂O₂ per hour, then the additive is provided in (a) so that the molar amount of M per hour is in the range of from 1.106×10⁻⁵ to 1.106×10⁻³.

As indicated above, organic solvent, an additive, water and optionally an olefin are provided in (a) for a first period of time P₁ to the epoxidation zone, so that at the end of P₁ the heterogeneous epoxidation catalyst has been contacted with a molar amount M per weight catalyst in the range of from 2500 to 4500 μmol M/kg catalyst—this equally applies for the additive being provided in at least one of (a), (a.1), (a.2), preferably in (a) or in (a.1) and (a.2) respectively. In some preferred embodiments of the start-up method, the additive is provided in at least one of (a), (a.1), (a.2), preferably in (a) or in (a.1) and (a.2) respectively, so that at the end of P₁ or P_{1.2} respectively, the heterogeneous epoxidation catalyst has been contacted with a molar amount M per weight catalyst in the range of from in the range of from 2600 to 4400 μmol M/kg catalyst, preferably in the range of from 2700 to 4300 μmol M/kg catalyst, more preferably in the range of from 2800 to 4200 μmol M/kg catalyst, more preferably in the range of from 2900 to 4100 μmol M/kg catalyst, more preferably in the range of from 3000 to 4000 μmol M/kg catalyst.

The additive is preferably continuously provided during the start-up so that there are no periods of time during start-up where no additive is provided.

In some preferred embodiments of the start-up method, the first period of time P₁ and/or the overall period of time of P_{1.1}.+P_{1.2} is a period of time in the range of from 15 to 120 minutes, preferably in the range of from 45 to 100 minutes, more preferably in the range of from 55 to 90 minutes, more preferably in the range of from 60 to 85 minutes, more preferably in the range of from 75 to 80 minutes.

In some preferred embodiments of the start-up method, the amount of olefin optionally provided in (a) and/or provided in (a.2) is lower than the amount intended to be provided in the normal run stage.

In some preferred embodiments, the start-up method further comprises
(b) after the period of time of P₁ or P_{1.2} respectively, providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst.

In some preferred embodiments of the start-up method, the amounts of olefin and of hydrogen peroxide provided in (b) are lower than the respective amounts intended to be provided in the normal run stage.

In some preferred embodiments of the start-up method, (b) comprises, preferably is,
(b′) after the period of time P₁ or P_{1.2} respectively, providing organic solvent, additive, water, olefin and hydrogen peroxide in initial amounts to the epoxidation zone for a period of time P_{2′}, so that a mixture is formed, which comprises organic solvent, additive, water olefin, and hydrogen peroxide, wherein the amounts of the hydrogen peroxide and of the olefin are lower than the amounts intended to be provided in the normal run stage, and increasing the amount of the hydrogen peroxide and the amount of the olefin over the period of time P_{2′} up to the amounts intended to be provided in the normal run stage, so that a mixture 2′ is formed, and contacting the mixture 2′ over P_{2′} under epoxidation reaction conditions in the epoxidation zone with the heterogeneous epoxidation catalyst.

In the mixture 2′, due to the increasing of the amount of the hydrogen peroxide and the amount of the olefin over the period of time P_{2′}, the concentrations of hydrogen peroxide and olefin increase over P_{2′}, wherein the amounts of olefin and of hydrogen peroxide provided in (b′) are lower than the respective amounts intended to be provided in the normal run stage—increasing the amounts of olefin and of hydrogen peroxide over the period of time P₂ goes until the amounts intended to be provided in the normal run stage are reached. The molar amounts of hydrogen peroxide and olefin initially provided in (b′) are preferably each in the range of from 1 to 50% of the amount intended to be used in the normal run stage. During P_{2′}, the amounts of hydrogen peroxide and of olefin are increased up to 100% of the amounts intended to be used in the normal run stage, wherein the increasing is carried out stepwise or continuously, so that the mixture formed in (b′) comprises increasing amounts of olefin and hydrogen peroxide compared to the amounts initially provided in (b′).

In some preferred embodiments of the start-up method, the olefin is provided in (a) or in (a.2) respectively in a molar ratio relative to the molar amount of hydrogen peroxide intended to be used in the normal run stage in the range of from 0.1:1 to 4, preferably in the range of from 0.1:1 to 4:1, or in the range of rom 2.1 to 4:1.

In some preferred embodiments of the start-up method, the olefin and the hydrogen peroxide are provided in (b) or in (b′) respectively so that the molar ratio of olefin:hydrogen peroxide in the second mixture 2 formed in (b) or in the mixture 2′ formed in (b′) respectively is in the range of from 1.1:1 to 5:1, preferably in the range of from 1.1:1 to 2:1 or in the range of from 3:1 to 5:1.

In the normal run stage of the process for preparing an olefin oxide, the olefin is preferably used in a molar excess compared to the hydrogen peroxide. The expression “the olefin is provided in (a.2) in a molar amount compared to the molar amount of hydrogen peroxide intended to be used in the normal run stage” means for the start-up method, that per mol of hydrogen peroxide intended to be used in the normal run stage, the exceeding molar amount of olefin in relation to a 1:1 ratio olefin:hydrogen peroxide is provided in (a.2) and thus contained in the mixture 1.2 formed in (a.2). In other words, the molar amount of olefin provided in (a.2) is (molar amount of hydrogen peroxide intended to be used in the normal run stage)−(molar amount of olefin intended to be used in the normal run stage). For example, in case the molar ratio olefin:hydrogen peroxide intended to be used in the normal run stage is 1.1:1, then the exceeding molar amount of olefin intended to be used in the normal run stage is 0.1. If, for example, it is intended for the normal run stage to use an aqueous H₂O₂ solution (40 weight-% H₂O₂) fed with a flow rate of 94 g/h in the normal run stage, i.e. 1.106 mol H₂O₂ per hour, then 0.22 mol propylene are fed per hour, meaning 9.26 g/h. In some preferred embodiments, the olefin is provided in (a) and/or in (a.2) in an amount in the range of from 0.25 to 1.5 kg olefin per kg of the catalyst and per hour

$$( \frac{kg propene}{kg catalyst hour} ) .$$

The point in time when step (a) or (a.1) respectively starts is t₁ respectively. The point in time when step (a.2) starts is t_{1.2} and the point in time when (b) or (b′) starts is t₂. The first period of time P₁ is thus the period of time between t₁ and t₂, i.e. P₁=t₂−t₁, while P_{1.1} is t_{1.2}−t₁ and P_{1.2} is t₂−t_{1.2}, and the second period of time P₂ as well as P₂ is/are the period of time between t₃ and t₂, i.e. P₂=t₃−t₂, P₂=t₃−t₂. When the amounts of all components (organic solvent, additive, water, olefin, and hydrogen peroxide) are as high as intended for the normal run stage, this indicates the beginning of the normal run stage, which happens at a point in time t₃.

As indicated above and also below, the amount of hydrogen peroxide provided in the normal run stage is adjusted so that the molar ratio of olefin:hydrogen peroxide in the reaction mixture formed is in the range of from 1.1:1 to 5:1, preferably in the range of from 1.1:1 to 2:1 or in the range of from 3:1 to 5:1. Therefore, in (b) or (b′) respectively preferably an excess of olefin compared to hydrogen peroxide is used, wherein in (a) or (a.2) respectively, only the exceeding molar amount of olefin compared to the molar amount of hydrogen peroxide—in relation to what is intended to be used in the normal run stage—is provided.

In some preferred embodiments of the start-up method (a.1) comprises
(a.1.1) providing organic solvent, additive and water for a period of time P_{1.1} to the epoxidation zone, so that a mixture 1.1 is formed, which is essentially free of hydrogen peroxide, and contacting the mixture 1.1 over P_{1.1} under epoxidation reaction conditions in the epoxidation zone with the heterogeneous epoxidation catalyst;
(a.1.2) removing during P_{1.1} an effluent stream from the epoxidation zone, the effluent stream comprising organic solvent, additive and water;
and/or
wherein (a.2) comprises
(a.2.1) providing organic solvent, additive, water and olefin for a period of time P_{1.2} to the epoxidation zone, so that a mixture 1.2 is formed, which is essentially free of hydrogen peroxide, and contacting the mixture 1.2 over P_{1.2} under epoxidation reaction conditions in the epoxidation zone with the heterogeneous epoxidation catalyst;
(a.2.2) removing during P_{1.2} an effluent stream from the epoxidation zone, the effluent stream comprising olefin, organic solvent, additive and water.

In some preferred embodiments of the start-up method (b) comprises
(b.1) after the period of time P₁ or P_{1.2} respectively providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst;
(b.2) removing during the respective period of time an effluent stream from the epoxidation zone, the effluent stream comprising olefin oxide, organic solvent, additive and water;
and/or
wherein (b′) comprises
(b′.1) after the second period of time P₁ or P_{1.2}, providing organic solvent, additive, water, olefin and hydrogen peroxide in initial amounts to the epoxidation zone for a period of time P_{2′}, so that a mixture is formed, which comprises organic solvent, additive, water, olefin, and hydrogen peroxide, wherein the amounts of the hydrogen peroxide and of the olefin are lower than the amounts intended to be provided in the normal run stage, and increasing the amount of the hydrogen peroxide and the amount of the olefin over the period of time P₂ up to the amounts intended to be provided in the normal run stage, so that a mixture 2′ is formed, and contacting the mixture 2′ over P₂ under epoxidation reaction conditions in the epoxidation zone with the heterogeneous epoxidation catalyst;
(b′.2) removing during the respective period of time an effluent stream from the epoxidation zone, the effluent stream comprising olefin oxide, organic solvent, additive and water.

### Additive in (a) Etc.

In some preferred embodiments of the start-up method, the additive is provided in any one of (a.2.1), (b), (b′), (b.1), (b′.1), in a molar ratio M:hydrogen peroxide in the range of from 1×10⁻⁵:1 to 1×10⁻³:1, based on the molar amount of hydrogen peroxide intended to be provided in the normal run stage. This means that the amount of additive is adjusted in anticipation of the amount of hydrogen peroxide intended to be provided in the normal run stage.

### Catalyst

In some preferred embodiments of the start-up method, the heterogeneous epoxidation catalyst comprises a zeolitic material having a framework structure comprising Si, O and Ti.

In some preferred embodiments of the start-up method, the zeolitic material comprises Ti in an amount in the range of from 0.2 to 5 weight-%, preferably in the range of from 0.5 to 4 weight-%, more preferably in the range of from 0.7 to 3 weight-%, calculated as elemental Ti and based on the total weight of the zeolitic material.

In some preferred embodiments of the start-up method, the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22 (S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O and Ti is a titanium zeolite having an MFI framework type, an MEL framework type, an MWW framework type, an MCM-22 (S) framework type, an MCM-56 framework type, an IEZ-MWW framework type, an MCM-36 framework type, an ITQ framework type, a BEA framework type, a MOR framework type, or a mixed structure of two or more of these framework types; more preferably the zeolitic material having a framework structure comprising Si, O and Ti is a titanium zeolite having an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).

In some preferred embodiments of the start-up method, the zeolitic material having a framework structure comprising Si, O and Ti is a titanium zeolite having an MFI framework type, which exhibits a type IV nitrogen adsorption/desorption isotherm determined at 77 K according to the method disclosed in DIN 66131 (July 1993).

In some preferred embodiments of the start-up method, the heterogeneous epoxidation catalyst further comprises a binder, which preferably comprises silicon dioxide.

In some preferred embodiments of the start-up method, the heterogeneous epoxidation catalyst is in the form of a molding, preferably in the form of a strand or a granule.

A strand has a cross section wherein the cross section preferably has a hexagonal, rectangular, quadratic, triangular, trilobe, oval, or circular shape. In some preferred embodiments, the molding has the form of a strand having a circular cross-section, which is more preferably formed by extrusion (cylindrical extrudate). A granule preferably comprises particles having a form selected from cylinder, sphere, trilobe (particle having a trilobe cross section), and mixed forms of two or more of these forms, wherein the granule comprises particles of one form as well as mixtures of particles having two or more of these forms. A “sphere” comprises an ideal sphere form but also spheroidal forms. A “cylinder” comprises forms having a high h which is larger than the radius of the circle area as well as forms having a high h which is smaller than the radius of the circle area (tablet form).

In some preferred embodiments of the start-up method, from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.

In some preferred embodiments of the start-up method, from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.

In some preferred embodiments of the start-up method, the heterogeneous epoxidation catalyst, preferably the molding, comprises the binder, calculated as SiO₂, in an amount in the range of from 1 to 95 weight-%, preferably in the range of from 3 to 70 weight-%, more preferably in the range of from 5 to 50 weight-%, more preferably in the range of from 10 to 30 weight-%, based on the total weight of the epoxidation catalyst, preferably based on the total weight of the molding and/or wherein the heterogeneous epoxidation catalyst, preferably the molding, comprises the zeolitic material in an amount in the range of from 5 to 99 weight-%, preferably in the range of from 30 to 97 weight-%, more preferably in the in the range of from 50 to 95 weight-%, more preferably in the range of from 70 to 90 weight-%, based on the total weight of the heterogeneous epoxidation catalyst, preferably based on the total weight of the molding.

As indicated above, organic solvent, an additive, water and optionally an olefin are provided in (a) for a first period of time P₁ to the epoxidation zone, so that at the end of P₁ the heterogeneous epoxidation catalyst has been contacted with a molar amount M per weight catalyst in the range of from 2500 to 4500 μmol M/kg catalyst—this equally applies for the additive being provided in at least one of (a), (a.1), (a.2), preferably in (a) or in (a.1) and (a.2) respectively; preferably the additive is provided in at least one of (a), (a.1), (a.2), preferably in (a) or in (a.1) and (a.2) respectively, so that at the end of P₁ or P_{1.2} respectively, the heterogeneous epoxidation catalyst has been contacted with a molar amount M per weight catalyst in the range of from in the range of from 2600 to 4400 μmol M/kg catalyst, more preferably in the range of from 2700 to 4300 μmol M/kg catalyst, more preferably in the range of from 2800 to 4200 μmol M/kg catalyst, more preferably in the range of from 2900 to 4100 μmol M/kg catalyst, more preferably in the range of from 3000 to 4000 μmol M/kg catalyst. In preferred embodiments of the start-up method, where the heterogeneous epoxidation catalyst comprises a zeolitic material having a framework structure comprising Si, O and Ti, providing in the range of from 2500 to 4500 μmol M/kg catalyst in at least one of (a), (a.1), (a.2), preferably in (a) or in (a. 1) and (a.2) respectively corresponds to providing in the range of from 31 to 58 μmol M/kg Ti contained in the catalyst; providing in the range of from in the range of from 2600 to 4400 μmol M/kg catalyst corresponds to providing in the range of from 32 to 56 μmol M/kg Ti contained in the catalyst; providing in the range of from 2700 to 4300 μmol M/kg catalyst corresponds to providing in the range of from 33 to 54 μmol M/kg Ti contained in the catalyst; providing in the range of from 2800 to 4200 μmol M/kg catalyst corresponds to providing in the range of from 35 to 53 μmol M/kg Ti contained in the catalyst; providing in the range of from 2900 to 4100 μmol M/kg catalyst corresponds to providing in the range of from 37 to 52 μmol M/kg Ti contained in the catalyst; and providing in the range of from 3000 to 4000 μmol M/kg catalyst corresponds to providing in the range of from 38 to 51 μmol M/kg Ti contained in the catalyst.

### Hydrogen Peroxide

In some preferred embodiments of the start-up method, the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019).

In some preferred embodiments of the start-up method, the hydrogen peroxide has a pH in the range of from 0 to 3.0, preferably in the range of from 0.1 to 2.5, more preferably in the range of from 0.5 to 2.3, determined with a pH sensitive glass electrode according to CEFIC PEROXYGENS H2O2 AM-7160 standard (2003).

In some preferred embodiments of the start-up method, the aqueous hydrogen peroxide solution comprises from 20 to 85 weight-%, preferably from 30 to 75 weight-%, more preferably from 40 to 70 weight-% of hydrogen peroxide, relative to the total weight of the aqueous hydrogen peroxide solution.

In some preferred embodiments of the start-up method, the hydrogen peroxide is obtained or obtainable from an anthraquinone process.

### Solvent

In some preferred embodiments of the start-up method, the organic solvent is an organic epoxidation solvent, preferably the organic solvent is selected from the group consisting of alcohol, acetonitrile, propionitrile and mixtures of two or more thereof; more preferably selected from the group consisting of alcohol, acetonitrile and mixtures of alcohol and acetonitrile; more preferably the organic solvent comprises at least an alcohol, wherein the alcohol is preferably a C₁ to C₅ mono alcohol or a mixture of two or more C₁ to C₅ alcohols, more preferably the alcohol comprises at least methanol, wherein the organic solvent more preferably comprises at least 90 weight-%, more preferably at least 95 weight-%, more preferably at least 98 weight-%, more preferably at least 99 weight-%, of methanol.

### Olefin (Oxide)

In some preferred embodiments of the start-up method, the olefin is a C₂-C₁₀ alkene, preferably a C₂-C₅ alkene, more preferably a C₂-C₄ alkene, more preferably ethylene or propylene, more preferably propylene. The olefin oxide, especially the olefin oxide removed from the epoxidation zone with the effluent stream comprising olefin oxide, water and organic solvent in (C) as described in more detail below, is a C₂-C₁₀ alkene oxide, preferably a C₂-C₅ alkene oxide, more preferably a C₂-C₄ alkene oxide, more preferably ethylene oxide or propylene oxide, more preferably propylene oxide.

### 2^{nd} Aspect—Process for Preparing an Olefin Oxide

A second aspect of the invention relates to a process for preparing an olefin oxide comprising a normal run stage and a start-up stage, wherein the normal run stage comprises
(A) providing olefin, hydrogen peroxide, additive water and organic solvent into an epoxidation zone comprising an heterogeneous epoxidation catalyst, so that a reaction mixture comprising olefin, hydrogen peroxide, additive, water and organic solvent is formed;
(B) subjecting the reaction mixture from (A) to epoxidation reaction conditions in the epoxidation zone, thereby obtaining a mixture comprising olefin oxide, additive, water and organic solvent;
(C) removing an effluent stream from the epoxidation zone, comprising olefin oxide, additive, water and organic solvent;
wherein the start-up stage comprises
(a) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst.

The first mixture 1 is essentially free of hydrogen peroxide. “Essentially free of hydrogen peroxide” regarding means that said first mixture 1 comprises less than 0.2 weight-%, preferably less than 0.1 weight-%, more preferably less than 0.05 weight-%, of hydrogen peroxide, based on the total weight of the first mixture 1. All details, preferred embodiments and alternative preferred embodiments indicated above in the section related to the first aspect apply also for the second aspect, especially for the start-up stage comprised in the process for preparing an olefin oxide.

In some preferred embodiments of the process for preparing an olefin oxide comprising a normal run stage and a start-up stage, the additive is selected from the group consisting of potassium salt of an inorganic acid, potassium salt of an organic acid, ammonia, ammonium salt of an inorganic acid, ammonium salt of an organic acid and mixtures of two or more thereof; wherein the additive is preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof, more preferably form the group consisting of potassium dihydrogen phosphate, dipotassium hydrogen phosphate, dipotassium etidronate, ammonia and mixtures of two or more thereof; wherein the additive more preferably comprises at least dipotassium etidronate; wherein more preferably in the range of from 95 to 100 weight-% of the additive are dipotassium etidronate.

In some preferred embodiments of the process for preparing an olefin oxide comprising a normal run stage and a start-up stage, the additive is in case of ammonia provided in pure form or in aqueous solution, wherein the additive is in case of being selected from the group consisting of potassium salt of an inorganic acid, potassium salt of an organic acid, ammonium salt of an inorganic acid, ammonium salt of an organic acid or any mixture comprising two or more salts of these acids or any mixture comprising ammonia and at least one salts of these acids provided in aqueous solution;

wherein M represents in case of a potassium salt K⁺, or, in case of ammonia or ammonium salts, M represents the sum of NH₄⁺ and NH₃.

Etidronic acid is a tetrabasic acid with pKs values of 1.35±0.08; 2.87; 7.03±0.01; 11.3. Each of the above-mentioned ammonia, potassium salts and ammonium salts, when in aqueous solution, dissociates to release or forms at least to some extend anion(s) and one or more cation(s). For example, ammonia forms to some extent NH₄⁺, ammonium salts dissociate to some extent to NH₄⁺ and one or more anion(s), or potassium salts dissociate to some extent to release K⁺ and one or more anion(s), the positive charge compensated by the appropriate equivalent of anion(s)). Regarding ammonia and ammonium salts, due to the dissociation equilibrium between NH₃ and NH₄⁺, the sum of NH₃ and NH₄⁺ has to be considered for M.

In some preferred embodiments of the process for preparing an olefin oxide comprising a normal run stage and a start-up stage, the additive is selected from the group consisting of potassium salt of an inorganic acid, potassium salt of an organic acid, ammonium salt of an inorganic acid, ammonium salt of an organic acid, a mixture comprising two or more of these acid salts and a mixture comprising ammonia and at least one of these acid salts, preferably from the group consisting of potassium salt of an inorganic acid, potassium salt of an organic acid, ammonium salt of an inorganic acid, ammonium salt of an organic acid, and a mixture comprising two or more of these acid salts, and is provided as an aqueous solution, wherein the aqueous solution of additive preferably comprises in the range of from 0.01 to 5 weight-%, more preferably in the range of from 0.5 to 3 weight-%, more preferably in the range of from 1 to 2 weight-%, of additive, based on the overall weight of the aqueous solution.

In some preferred embodiments of the process for preparing an olefin oxide comprising a normal run stage and a start-up stage, the additive is ammonia, which is provided in pure form or in aqueous solution, wherein the aqueous solution of ammonia preferably comprises in the range of from 0.01 to 32 weight-% of ammonia, based on the overall weight of the aqueous solution.

“Pure form” regarding ammonia means that a composition comprising ammonia is used, which comprises in the range of from 95 to 100 weight-%, preferably in the range of from 96 to 100 weight-%, more preferably in the range of from 97 to 100 weight-%, more preferably in the range of from 98 to 100 weight-%, more preferably in the range of from 99 to 100 weight-%, more preferably in the range of from 99.5 to 100 weight-%, of ammonia, based on the overall weight of the composition being 100 weight-%, the remainder up to 100 weight-% preferably being water.

In some preferred embodiments of the process for preparing an olefin oxide comprising a normal run stage and a start-up stage, (a) comprises
(a.1) providing organic solvent, an additive and water for a period of time P_{1.1} to the epoxidation zone, so that a mixture 1.1 is formed, which comprises organic solvent, additive and water and contacting the mixture 1.1 over P_{1.1} under epoxidation reaction conditions in the epoxidation zone with the heterogeneous epoxidation catalyst;
(a.2) providing organic solvent, additive, water and olefin for a second period of time P_{1.2} to the epoxidation zone, so that a mixture 1.2 is formed, which comprises organic solvent, additive, water and olefin, and contacting the second mixture 1.2 over P_{1.2} under epoxidation reaction conditions in the epoxidation zone with the heterogeneous epoxidation catalyst.

Preferably, the mixture 1.1 formed in (a. 1) is essentially free of olefin and essential free of hydrogen peroxide. The mixture 1.2 formed in (a.2) is essentially free of hydrogen peroxide “Essentially free of hydrogen peroxide” regarding the mixture 1.1 in (a.1) and the mixture 1.2 in (a.2) means that said mixture comprises less than 0.2 weight-%, preferably less than 0.1 weight-%, more preferably less than 0.05 weight-%, of hydrogen peroxide, based on the total weight of the mixture. “Essentially free of olefin” regarding the mixture 1.1 in (a. 1) means that said mixture comprises less than 0.2 weight-%, preferably less than 0.1 weight-%, more preferably less than 0.05 weight-%, of olefin, based on the total weight of the mixture.

In some preferred embodiments of the process for preparing an olefin oxide comprising a normal run stage and a start-up stage, the additive is provided in at least one of (a), (a.1), (a.2), preferably in (a) or in (a.1), (a.2) respectively, in a molar ratio M:hydrogen peroxide intended to be provided in the normal run stage in the range of from 1×10⁻⁵:1 to 1×10⁻³:1.

In some preferred embodiments of the process for preparing an olefin oxide comprising a normal run stage and a start-up stage, the additive is provided in at least one of (a), (a.1), (a.2), preferably in (a) or in (a.1) and (a.2) respectively, so that at the end of P₁ or P_{1.2} respectively, the heterogeneous epoxidation catalyst has been contacted with a molar amount M per weight catalyst in the range of from in the range of from 2600 to 4400 μmol M/kg catalyst, preferably in the range of from 2700 to 4300 μmol M/kg catalyst, more preferably in the range of from 2800 to 4200 μmol M/kg catalyst, more preferably in the range of from 2900 to 4100 μmol M/kg catalyst, more preferably in the range of from 3000 to 4000 μmol M/kg catalyst.

The additive is preferably continuously provided during the start-up so that there are no periods of time during start-up where no additive is provided.

In some preferred embodiments of the process for preparing an olefin oxide comprising a normal run stage and a start-up stage, the first period of time P₁ and/or the overall period of time of P_{1.1}.+P_{1.2} is a period of time in the range of from 15 to 120 minutes, preferably in the range of from 45 to 100 minutes, more preferably in the range of from 55 to 90 minutes, more preferably in the range of from 60 to 85 minutes, more preferably in the range of from 75 to 80 minutes.

In some preferred embodiments of the process for preparing an olefin oxide comprising a normal run stage and a start-up stage, the amount of olefin optionally provided in (a) and/or provided in (a.2) is lower than the amount intended to be provided in the normal run stage.

In some preferred embodiments, the process for preparing an olefin oxide comprising a normal run stage and a start-up stage further comprises
(b) after the period of time of P₁ or P_{1.2} respectively, providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst.

In some preferred embodiments of the process for preparing an olefin oxide comprising a normal run stage and a start-up stage, the amounts of olefin and of hydrogen peroxide provided in (b) are lower than the respective amounts intended to be provided in the normal run stage.

In some preferred embodiments of the process for preparing an olefin oxide comprising a normal run stage and a start-up stage (b) comprises, preferably is,
(b′) after the period of time P₁ or P_{1.2} respectively, providing organic solvent, additive, water, olefin and hydrogen peroxide in initial amounts to the epoxidation zone for a period of time P_{2′}, so that a mixture is formed, which comprises organic solvent, additive, water olefin, and hydrogen peroxide, wherein the amounts of the hydrogen peroxide and of the olefin are lower than the amounts intended to be provided in the normal run stage, and increasing the amount of the hydrogen peroxide and the amount of the olefin over the period of time P_{2′} up to the amounts intended to be provided in the normal run stage, so that a mixture 2′ is formed, and contacting the mixture 2′ over P_{2′} under epoxidation reaction conditions in the epoxidation zone with the heterogeneous epoxidation catalyst.

In the mixture 2′, due to the increasing of the amount of the hydrogen peroxide and the amount of the olefin over the period of time P_{2′}, the concentrations of hydrogen peroxide and olefin increase over P_{2′}, wherein the amounts of olefin and of hydrogen peroxide provided in (b′) are lower than the respective amounts intended to be provided in the normal run stage—increasing the amounts of olefin and of hydrogen peroxide over the period of time P₂ goes until the amounts intended to be provided in the normal run stage are reached. The molar amounts of hydrogen peroxide and olefin initially provided in (b′) are preferably each in the range of from 1 to 50% of the amount intended to be used in the normal run stage. During P_{2′}, the amounts of hydrogen peroxide and of olefin are increased up to 100% of the amounts intended to be used in the normal run stage, wherein the increasing is carried out stepwise or continuously, so that the mixture formed in (b′) comprises increasing amounts of olefin and hydrogen peroxide compared to the amounts initially provided in (b′).

In some preferred embodiments of the process for preparing an olefin oxide comprising a normal run stage and a start-up stage, the olefin is provided in (a) or in (a.2) respectively in a molar ratio relative to the molar amount of hydrogen peroxide intended to be used in the normal run stage in the range of from 0.1:1 to 4, preferably in the range of from 0.1:1 to 4:1, or in the range of rom 2.1 to 4:1.

In some preferred embodiments of the process for preparing an olefin oxide comprising a normal run stage and a start-up stage, the olefin and the hydrogen peroxide are provided in (b) or in (b′) respectively so that the molar ratio of olefin:hydrogen peroxide in the second mixture 2 formed in (b) or in the mixture 2′ formed in (b′) respectively is in the range of from 1.1:1 to 5:1, preferably in the range of from 1.1:1 to 2:1 or in the range of from 3:1 to 5:1.

In the normal run stage of the process for preparing an olefin oxide, the olefin is preferably used in a molar excess compared to the hydrogen peroxide. The expression “the olefin is provided in (a.2) in a molar amount compared to the molar amount of hydrogen peroxide intended to be used in the normal run stage” means for the start-up method, that per mol of hydrogen peroxide intended to be used in the normal run stage, the exceeding molar amount of olefin in relation to a 1:1 ratio olefin:hydrogen peroxide is provided in (a.2) and thus contained in the mixture 1.2 formed in (a.2). In other words, the molar amount of olefin provided in (a.2) is (molar amount of hydrogen peroxide intended to be used in the normal run stage)−(molar amount of olefin intended to be used in the normal run stage). For example, in case the molar ratio olefin:hydrogen peroxide intended to be used in the normal run stage is 1.1:1, then the exceeding molar amount of olefin intended to be used in the normal run stage is 0.1. If, for example, it is intended for the normal run stage to use an aqueous H₂O₂ solution (40 weight-% H₂O₂) fed with a flow rate of 94 g/h in the normal run stage, i.e. 1.106 mol H₂O₂ per hour, then 0.22 mol propylene are fed per hour, meaning 9.26 g/h. In some preferred embodiments, the olefin is provided in (a) and/or in (a.2) in an amount in the range of from 0.25 to 1.5 kg olefin per kg of the catalyst and per hour

$$( \frac{kg propene}{kg catalyst hour} ) .$$

The point in time when step (a) or (a.1) respectively starts is t₁ respectively. The point in time when step (a.2) starts is t_{1.2} and the point in time when (b) or (b′) starts is t₂. The first period of time P₁ is thus the period of time between t₁ and t₂, i.e. P₁=t₂−t₁, while P_{1.1} is t_{1.2}−t₁ and P_{1.2} is t₂−t_{1.2}, and the second period of time P₂ as well as P₂ is/are the period of time between t₃ and t₂, i.e. P₂=t₃−t₂, P₂=t₃−t₂. When the amounts of all components (organic solvent, additive, water, olefin, and hydrogen peroxide) are as high as intended for the normal run stage, this indicates the beginning of the normal run stage, which happens at a point in time t₃.

As indicated above and also below, the amount of hydrogen peroxide provided in the normal run stage is adjusted so that the molar ratio of olefin:hydrogen peroxide in the reaction mixture formed is in the range of from 1.1:1 to 5:1, preferably in the range of from 1.1:1 to 2:1 or in the range of from 3:1 to 5:1. Therefore, in (b) or (b′) respectively preferably an excess of olefin compared to hydrogen peroxide is used, wherein in (a) or (a.2) respectively, only the exceeding molar amount of olefin compared to the molar amount of hydrogen peroxide—in relation to what is intended to be used in the normal run stage—is provided.

In some preferred embodiments of the process for preparing an olefin oxide comprising a normal run stage and a start-up stage, (a.1) comprises
(a.1.1) providing organic solvent, additive and water for a period of time P_{1.1} to the epoxidation zone, so that a mixture 1.1 is formed, which is essentially free of hydrogen peroxide, and contacting the mixture 1.1 over P_{1.1} under epoxidation reaction conditions in the epoxidation zone with the heterogeneous epoxidation catalyst;
(a.1.2) removing during P_{1.1} an effluent stream from the epoxidation zone, the effluent stream comprising organic solvent, additive and water;
and/or
wherein (a.2) comprises
(a.2.1) providing organic solvent, additive, water and olefin for a period of time P_{1.2} to the epoxidation zone, so that a mixture 1.2 is formed, which is essentially free of hydrogen peroxide, and contacting the mixture 1.2 over P_{1.2} under epoxidation reaction conditions in the epoxidation zone with the heterogeneous epoxidation catalyst;
(a.2.2) removing during P_{1.2} an effluent stream from the epoxidation zone, the effluent stream comprising olefin, organic solvent, additive and water.

In some preferred embodiments of the process for preparing an olefin oxide comprising a normal run stage and a start-up stage (b) comprises
(b.1) after the period of time P₁ or P_{1.2} respectively providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst;
(b.2) removing during the respective period of time an effluent stream from the epoxidation zone, the effluent stream comprising olefin oxide, organic solvent, additive and water;
and/or
wherein (b′) comprises
(b′.1) after the second period of time P₁ or P_{1.2}, providing organic solvent, additive, water, olefin and hydrogen peroxide in initial amounts to the epoxidation zone for a period of time P_{2′}, so that a mixture is formed, which comprises organic solvent, additive, water, olefin, and hydrogen peroxide, wherein the amounts of the hydrogen peroxide and of the olefin are lower than the amounts intended to be provided in the normal run stage, and increasing the amount of the hydrogen peroxide and the amount of the olefin over the period of time P₂ up to the amounts intended to be provided in the normal run stage, so that a mixture 2′ is formed, and contacting the mixture 2′ over P₂ under epoxidation reaction conditions in the epoxidation zone with the heterogeneous epoxidation catalyst;
(b′.2) removing during the respective period of time an effluent stream from the epoxidation zone, the effluent stream comprising olefin oxide, organic solvent, additive and water.

### Additive in (a) Etc.

In some preferred embodiments of the process for preparing an olefin oxide comprising a normal run stage and a start-up stage, the additive is provided in any one of (a.2.1), (b), (b′), (b.1), (b′.1), in a molar ratio M:hydrogen peroxide in the range of from 1×10⁻⁵:1 to 1×10⁻³:1, based on the molar amount of hydrogen peroxide intended to be provided in the normal run stage. This means that the amount of additive is adjusted in anticipation of the amount of hydrogen peroxide intended to be provided in the normal run stage.

### Catalyst

In some preferred embodiments of the process for preparing an olefin oxide comprising a normal run stage and a start-up stage, the heterogeneous epoxidation catalyst comprises a zeolitic material having a framework structure comprising Si, O and Ti.

In some preferred embodiments of the process for preparing an olefin oxide comprising a normal run stage and a start-up stage, the zeolitic material comprises Ti in an amount in the range of from 0.2 to 5 weight-%, preferably in the range of from 0.5 to 4 weight-%, more preferably in the range of from 0.7 to 3 weight-%, calculated as elemental Ti and based on the total weight of the zeolitic material.

In some preferred embodiments of the process for preparing an olefin oxide comprising a normal run stage and a start-up stage, the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22 (S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably the zeolitic material having a framework structure comprising Si, O and Ti is a titanium zeolite having an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).

In some preferred embodiments of the process for preparing an olefin oxide comprising a normal run stage and a start-up stage, zeolitic material having a framework structure comprising Si, O and Ti is a titanium zeolite having an MFI framework type, which exhibits a type IV nitrogen adsorption/desorption isotherm determined at 77 K according to the method disclosed in DIN 66131 (July 1993).

In some preferred embodiments of the process for preparing an olefin oxide comprising a normal run stage and a start-up stage, the heterogeneous epoxidation catalyst further comprises a binder, which preferably comprises silicon dioxide.

In some preferred embodiments of the process for preparing an olefin oxide comprising a normal run stage and a start-up stage, the heterogeneous epoxidation catalyst is in the form of a molding, preferably in the form of a strand or a granule.

A strand has a cross section wherein the cross section preferably has a hexagonal, rectangular, quadratic, triangular, trilobe, oval, or circular shape. In some preferred embodiments, the molding has the form of a strand having a circular cross-section, which is more preferably formed by extrusion (cylindrical extrudate). A granule preferably comprises particles having a form selected from cylinder, sphere, trilobe (particle having a trilobe cross section), and mixed forms of two or more of these forms, wherein the granule comprises particles of one form as well as mixtures of particles having two or more of these forms. A “sphere” comprises an ideal sphere form but also spheroidal forms. A “cylinder” comprises forms having a high h which is larger than the radius of the circle area as well as forms having a high h which is smaller than the radius of the circle area (tablet form).

In some preferred embodiments of the process for preparing an olefin oxide comprising a normal run stage and a start-up stage, from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.

In some preferred embodiments of the process for preparing an olefin oxide comprising a normal run stage and a start-up stage, from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.

In some preferred embodiments of the process for preparing an olefin oxide comprising a normal run stage and a start-up stage, the heterogeneous epoxidation catalyst, preferably the molding, comprises the binder, calculated as SiO₂, in an amount in the range of from 1 to 95 weight-%, preferably in the range of from 3 to 70 weight-%, more preferably in the range of from 5 to 50 weight-%, more preferably in the range of from 10 to 30 weight-%, based on the total weight of the epoxidation catalyst, preferably based on the total weight of the molding and/or wherein the heterogeneous epoxidation catalyst, preferably the molding, comprises the zeolitic material in an amount in the range of from 5 to 99 weight-%, preferably in the range of from 30 to 97 weight-%, more preferably in the in the range of from 50 to 95 weight-%, more preferably in the range of from 70 to 90 weight-%, based on the total weight of the heterogeneous epoxidation catalyst, preferably based on the total weight of the molding.

As indicated above, organic solvent, an additive, water and optionally an olefin are provided in (a) for a first period of time P₁ to the epoxidation zone, so that at the end of P₁ the heterogeneous epoxidation catalyst has been contacted with a molar amount M per weight catalyst in the range of from 2500 to 4500 μmol M/kg catalyst—this equally applies for the additive being provided in at least one of (a), (a.1), (a.2), preferably in (a) or in (a.1) and (a.2) respectively; preferably the additive is provided in at least one of (a), (a. 1), (a.2), preferably in (a) or in (a. 1) and (a.2) respectively, so that at the end of P₁ or P_{1.2} respectively, the heterogeneous epoxidation catalyst has been contacted with a molar amount M per weight catalyst in the range of from in the range of from 2600 to 4400 μmol M/kg catalyst, more preferably in the range of from 2700 to 4300 μmol M/kg catalyst, more preferably in the range of from 2800 to 4200 μmol M/kg catalyst, more preferably in the range of from 2900 to 4100 μmol M/kg catalyst, more preferably in the range of from 3000 to 4000 μmol M/kg catalyst. In preferred embodiments of the start-up method, where the heterogeneous epoxidation catalyst comprises a zeolitic material having a framework structure comprising Si, O and Ti, providing in the range of from 2500 to 4500 μmol M/kg catalyst in at least one of (a), (a. 1), (a.2), preferably in (a) or in (a.1) and (a.2) respectively corresponds to providing in the range of from 31 to 58 μmol M/kg Ti contained in the catalyst; providing in the range of from in the range of from 2600 to 4400 μmol M/kg catalyst corresponds to providing in the range of from 32 to 56 μmol M/kg Ti contained in the catalyst; providing in the range of from 2700 to 4300 μmol M/kg catalyst corresponds to providing in the range of from 33 to 54 μmol M/kg Ti contained in the catalyst; providing in the range of from 2800 to 4200 μmol M/kg catalyst corresponds to providing in the range of from 35 to 53 μmol M/kg Ti contained in the catalyst; providing in the range of from 2900 to 4100 μmol M/kg catalyst corresponds to providing in the range of from 37 to 52 μmol M/kg Ti contained in the catalyst; and providing in the range of from 3000 to 4000 μmol M/kg catalyst corresponds to providing in the range of from 38 to 51 μmol M/kg Ti contained in the catalyst.

### Hydrogen Peroxide

In some preferred embodiments of the process for preparing an olefin oxide comprising a normal run stage and a start-up stage, the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019).

In some preferred embodiments of the process for preparing an olefin oxide comprising a normal run stage and a start-up stage, the hydrogen peroxide has a pH in the range of from 0 to 3.0, preferably in the range of from 0.1 to 2.5, more preferably in the range of from 0.5 to 2.3, determined with a pH sensitive glass electrode according to CEFIC PEROXYGEN H2O2 AM-7160 standard (2003).

In some preferred embodiments of the process for preparing an olefin oxide comprising a normal run stage and a start-up stage, the aqueous hydrogen peroxide solution comprises from 20 to 85 weight-%, preferably from 30 to 75 weight-%, more preferably from 40 to 70 weight-% of hydrogen peroxide, relative to the total weight of the aqueous hydrogen peroxide solution.

In some preferred embodiments of the process for preparing an olefin oxide comprising a normal run stage and a start-up stage, the hydrogen peroxide is obtained or obtainable from an anthraquinone process.

### Solvent

In some preferred embodiments of the process for preparing an olefin oxide comprising a normal run stage and a start-up stage, the organic solvent is an organic epoxidation solvent, preferably the organic solvent is selected from the group consisting of alcohol, acetonitrile, propionitrile and mixtures of two or more thereof; more preferably selected from the group consisting of alcohol, acetonitrile and mixtures of alcohol and acetonitrile; more preferably the organic solvent comprises at least an alcohol, wherein the alcohol is preferably a C₁ to C₅ mono alcohol or a mixture of two or more C₁ to C₅ alcohols, more preferably the alcohol comprises at least methanol, wherein the organic solvent more preferably comprises at least 90 weight-%, more preferably at least 95 weight-%, more preferably at least 98 weight-%, more preferably at least 99 weight-%, of methanol.

### Olefin (Oxide)

In some preferred embodiments of the process for preparing an olefin oxide comprising a normal run stage and a start-up stage, the olefin is a C₂-C₁₀ alkene, preferably a C₂-C₅ alkene, more preferably a C₂-C₄ alkene, more preferably ethylene or propylene, more preferably propylene. The olefin oxide, especially the olefin oxide removed from the epoxidation zone with the effluent stream comprising olefin oxide, water and organic solvent in (C) is a C₂-C₁₀ alkene oxide, preferably a C₂-C₅ alkene oxide, more preferably a C₂-C₄ alkene oxide, more preferably ethylene oxide or propylene oxide, more preferably propylene oxide.

In some preferred embodiments of the process for preparing an olefin oxide comprising a normal run stage and a start-up stage, the epoxidation in (B) is carried out at an absolute pressure in the reaction zones in the range of from 0.5 to 5.0 MPa, preferably in the range of from 1.5 to 3.0 MPa, more preferably in the range of from 1.8 to 2.8 MPa.

In some preferred embodiments of the process for preparing an olefin oxide comprising a normal run stage and a start-up stage, the epoxidation in (B) is carried out at a temperature in the reaction zones in the range of from 20 to 75° C., preferably in the range of from 25 to 75° C., more preferably in the range of from 28 to 70° C., more preferably in the range of from 30 to 65° C.

In some preferred embodiments of the process for preparing an olefin oxide comprising a normal run stage and a start-up stage, the molar ratio of olefin:hydrogen peroxide (w/w) in the reaction mixture formed in (A) is in the range of from 1.1:1 to 5:1, preferably in the range of from 1.1:1 to 2:1 or in the range of from 3:1 to 5:1.

In some preferred embodiments of the process for preparing an olefin oxide comprising a normal run stage and a start-up stage, the weight ratio of organic solvent:hydrogen peroxide (w/w) in the reaction mixture formed in (A) is in the range of from 15:1 to 5:1, preferably in the range of from 12:1 to 6:1, more preferably in the range of from 12:1 to 8.5:1 or in the range of from 8:1 to 6:1.

In some preferred embodiments of the process for preparing an olefin oxide comprising a normal run stage and a start-up stage, the weight ratio of organic solvent:olefin (w/w) in the reaction mixture formed in (A) is in the range of from 10:1 to 1:0.1, preferably in the range of from 9:1 to 1:1, more preferably in the range of from 7:1 to 4:1 or in the range of from 1.5:1 to 1:1.

In some preferred embodiments of the process for preparing an olefin oxide comprising a normal run stage and a start-up stage, an additive is provided in (A), wherein the additive is selected from the group consisting of potassium salt of an inorganic acid, potassium salt of an organic acid, ammonia, ammonium salt of an inorganic acid, ammonium salt of an organic acid and mixtures of two or more thereof;

wherein the additive is preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof, more preferably form the group consisting of potassium dihydrogen phosphate, dipotassium hydrogen phosphate, dipotassium etidronate, ammonia and mixtures of two or more thereof; wherein the additive more preferably comprises at least dipotassium etidronate; wherein more preferably in the range of from 95 to 100 weight-% of the additive are dipotassium etidronate.

In some preferred embodiments of the process for preparing an olefin oxide comprising a normal run stage and a start-up stage, the additive is in case of ammonia provided in pure form or in aqueous solution, wherein the additive is in case of being selected from the group consisting of potassium salt of an inorganic acid, potassium salt of an organic acid, ammonium salt of an inorganic acid, ammonium salt of an organic acid or any mixture comprising two or more thereof or any mixture comprising ammonia and at least one thereof provided in aqueous solution; wherein the additive in aqueous solution releases or forms one or more cation(s) M, wherein M represents in case of a potassium salt K⁺, or, in case of ammonia or ammonium salts, M represents the sum of NH₄⁺ and NH₃.

In some preferred embodiments of the process for preparing an olefin oxide comprising a normal run stage and a start-up stage, the aqueous solution of additive comprises in the range of from 0.01 to 5 weight-%, preferably in the range of from 0.5 to 3 weight-%, more preferably in the range of from 1 to 2 weight-%, of additive, based on the overall weight of the aqueous solution.

In some preferred embodiments of the process for preparing an olefin oxide comprising a normal run stage and a start-up stage, the additive is preferably pre-mixed with the organic solvent or with the hydrogen peroxide, more preferably pre-mixed with the organic solvent, before being provided to the epoxidation zone in (A).

In some preferred embodiments of the process for preparing an olefin oxide comprising a normal run stage and a start-up stage, the additive is provided in (A) in a molar ratio M:hydrogen peroxide in the range of from 1×10⁻⁵:1 to 1×10³:1.

In some preferred embodiments of the process for preparing an olefin oxide comprising a normal run stage and a start-up stage, the epoxidation reaction conditions according to (B) comprise fixed bed conditions.

In some preferred embodiments of the process for preparing an olefin oxide comprising a normal run stage and a start-up stage, the epoxidation reaction conditions according to (B) comprise trickle bed conditions.

The disclosure above for the process of the second aspect equally applies for the steps such as (A), (B), (C) of the normal run stage and for the steps such as (a), (b) of the start-up stage.

### 3^{rd} Aspect—Olefin Oxide

In a third aspect, the invention is directed to an olefin oxide, preferably propylene oxide, obtained or obtainable from the process of the second aspect.

The present invention is further illustrated by the following embodiments and combinations of embodiments as indicated by the respective dependencies and back-references. In particular, it is noted that in each instance where a range of embodiments is mentioned, for example in the context of a term such as “The . . . of any of embodiments 1 to 4”, every embodiment in this range is meant to be explicitly disclosed for the skilled person, i.e. the wording of this term is to be understood by the skilled person as being synonymous to “The . . . of any of embodiments 1, 2, 3, and 4”.
1. A start-up method for a process for preparing an olefin oxide comprising a normal run stage, wherein the normal run stage comprises providing olefin, hydrogen peroxide, additive, water and organic solvent into an epoxidation zone comprising an heterogeneous epoxidation catalyst, so that a reaction mixture comprising olefin, hydrogen peroxide, additive, water and organic solvent is formed and subjecting the reaction mixture to epoxidation reaction conditions in the epoxidation zone, thereby obtaining a mixture comprising olefin oxide and organic solvent;
   wherein the start-up method comprises
   (b) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst;
   wherein the additive releases or forms one or more cation(s) M, wherein at the end of P₁ the heterogeneous epoxidation catalyst has been contacted with a molar amount M per weight catalyst in the range of from 2500 to 4500 μmol M/kg catalyst.
2. The start-up method of embodiment 1, wherein the additive is selected from the group consisting of potassium salt of an inorganic acid, potassium salt of an organic acid, ammonia, ammonium salt of an inorganic acid, ammonium salt of an organic acid and mixtures of two or more thereof;
   wherein the additive is preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof,
   more preferably form the group consisting of potassium dihydrogen phosphate, dipotassium hydrogen phosphate, dipotassium etidronate, ammonia and mixtures of two or more thereof;
   wherein the additive more preferably comprises at least dipotassium etidronate; wherein more preferably in the range of from 95 to 100 weight-% of the additive are dipotassium etidronate.
3. The start-up method of embodiment 1 or 2, wherein the additive is in case of ammonia provided in pure form or in aqueous solution, wherein the additive is in case of being selected from the group consisting of potassium salt of an inorganic acid, potassium salt of an organic acid, ammonium salt of an inorganic acid, ammonium salt of an organic acid or any mixture comprising two or more salts of these acids or any mixture comprising ammonia and at least one salts of these acids provided in aqueous solution;
   wherein M represents in case of a potassium salt K⁺, or, in case of ammonia or ammonium salts, M represents the sum of NH₄⁺ and NH₃.
4. The start-up method of embodiment 3, wherein the additive is selected from the group consisting of potassium salt of an inorganic acid, potassium salt of an organic acid, ammonium salt of an inorganic acid, ammonium salt of an organic acid, a mixture comprising two or more of these acid salts and a mixture comprising ammonia and at least one of these acid salts, preferably from the group consisting of potassium salt of an inorganic acid, potassium salt of an organic acid, ammonium salt of an inorganic acid, ammonium salt of an organic acid, and a mixture comprising two or more of these acid salts, and is provided as an aqueous solution, wherein the aqueous solution of additive preferably comprises in the range of from 0.01 to 5 weight-%, more preferably in the range of from 0.5 to 3 weight-%, more preferably in the range of from 1 to 2 weight-%, of additive, based on the overall weight of the aqueous solution.
5. The start-up method of embodiment 3, wherein the additive is ammonia, which is provided in pure form or in aqueous solution, wherein the aqueous solution of ammonia preferably comprises in the range of from 0.01 to 32 weight-% of ammonia, based on the overall weight of the aqueous solution.
6. The start-up method of any one of embodiments 1 to 5, wherein (a) comprises
   (a.1) providing organic solvent, an additive and water for a period of time P_{1.1} to the epoxidation zone, so that a mixture 1.1 is formed, which comprises organic solvent, additive and water and contacting the mixture 1.1 over P_{1.1} under epoxidation reaction conditions in the epoxidation zone with the heterogeneous epoxidation catalyst;
(a.2) providing organic solvent, additive, water and olefin for a second period of time P_{1.2} to the epoxidation zone, so that a mixture 1.2 is formed, which comprises organic solvent, additive, water and olefin, and contacting the second mixture 1.2 over P_{1.2} under epoxidation reaction conditions in the epoxidation zone with the heterogeneous epoxidation catalyst.
7. The start-up method of any one of embodiments 1 to 6, wherein the additive is provided in at least one of (a), (a.1), (a.2), preferably in (a) or in (a.1), (a.2) respectively, in a molar ratio M:hydrogen peroxide intended to be provided in the normal run stage in the range of from 1×10⁻⁵:1 to 1×10⁻³:1.
8. The start-up method of any one of embodiments 1 to 7, wherein the additive is provided in at least one of (a), (a.1), (a.2), preferably in (a) or in (a.1) and (a.2) respectively, so that at the end of P₁ or P_{1.2} respectively, the heterogeneous epoxidation catalyst has been contacted with a molar amount M per weight catalyst in the range of from in the range of from 2600 to 4400 μmol M/kg catalyst, preferably in the range of from 2700 to 4300 μmol M/kg catalyst, more preferably in the range of from 2800 to 4200 μmol M/kg catalyst, more preferably in the range of from 2900 to 4100 μmol M/kg catalyst, more preferably in the range of from 3000 to 4000 μmol M/kg catalyst.
9. The start-up method of any one of embodiments 1 to 8, wherein the first period of time P₁ and/or the overall period of time of P_{1.1}.+P₁₂ is a period of time in the range of from 15 to 120 minutes, preferably in the range of from 45 to 100 minutes, more preferably in the range of from 55 to 90 minutes, more preferably in the range of from 60 to 85 minutes, more preferably in the range of from 75 to 80 minutes.
10. The start-up method of any one of embodiments 6 to 9, wherein the amount of olefin optionally provided in (a) and/or provided in (a.2) is lower than the amount intended to be provided in the normal run stage.
11. The start-up method of any one of embodiments 1 to 10, further comprising
   (b) after the period of time of P₁ or P_{1.2} respectively, providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst.
12. The start-up method of embodiment 11, wherein the amounts of olefin and of hydrogen peroxide provided in (b) are lower than the respective amounts intended to be provided in the normal run stage.
13. The start-up method of embodiment 11 or 12, wherein (b) comprises, preferably is,
   (b′) after the period of time P₁ or P_{1.2} respectively, providing organic solvent, additive, water, olefin and hydrogen peroxide in initial amounts to the epoxidation zone for a period of time P_{2′}, so that a mixture is formed, which comprises organic solvent, additive, water olefin, and hydrogen peroxide, wherein the amounts of the hydrogen peroxide and of the olefin are lower than the amounts intended to be provided in the normal run stage, and
      increasing the amount of the hydrogen peroxide and the amount of the olefin over the period of time P_{2′} up to the amounts intended to be provided in the normal run stage, so that a mixture 2′ is formed, and contacting the mixture 2′ over P_{2′} under epoxidation reaction conditions in the epoxidation zone with the heterogeneous epoxidation catalyst.
14. The start-up method of any one of embodiments 1 to 13, wherein the olefin is provided in (a) or in (a.2) respectively in a molar ratio relative to the molar amount of hydrogen peroxide intended to be used in the normal run stage in the range of from 0.1:1 to 4, preferably in the range of from 0.1:1 to 4:1, or in the range of rom 2.1 to 4:1.
15. The start-up method of any one of embodiments 11 to 14, wherein the olefin and the hydrogen peroxide are provided in (b) or in (b′) respectively so that the molar ratio of olefin:hydrogen peroxide in the second mixture 2 formed in (b) or in the mixture 2′ formed in (b′) respectively is in the range of from 1.1:1 to 5:1, preferably in the range of from 1.1:1 to 2:1 or in the range of from 3:1 to 5:1.
16. The start-up method of any one of embodiments 6 to 15, wherein (a.1) comprises
   (a.1.1) providing organic solvent, additive and water for a period of time P_{1.1} to the epoxidation zone, so that a mixture 1.1 is formed, which is essentially free of hydrogen peroxide, and contacting the mixture 1.1 over P_{1.1} under epoxidation reaction conditions in the epoxidation zone with the heterogeneous epoxidation catalyst;
   (a.1.2) removing during P_{1.1} an effluent stream from the epoxidation zone, the effluent stream comprising organic solvent, additive and water;
   and/or
   wherein (a.2) comprises
   (a.2.1) providing organic solvent, additive, water and olefin for a period of time P_{1.2} to the epoxidation zone, so that a mixture 1.2 is formed, which is essentially free of hydrogen peroxide, and contacting the mixture 1.2 over P_{1.2} under epoxidation reaction conditions in the epoxidation zone with the heterogeneous epoxidation catalyst;
   (a.2.2) removing during P_{1.2} an effluent stream from the epoxidation zone, the effluent stream comprising olefin, organic solvent, additive and water.
17. The start-up method of any one of embodiments 13 to 16, wherein (b) comprises
   (b.1) after the period of time P₁ or P_{1.2} respectively providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst;
   (b.2) removing during the respective period of time an effluent stream from the epoxidation zone, the effluent stream comprising olefin oxide, organic solvent, additive and water;
   and/or
   wherein (b′) comprises
   (b′.1) after the second period of time P₁ or P_{1.2}, providing organic solvent, additive, water, olefin and hydrogen peroxide in initial amounts to the epoxidation zone for a period of time P_{2′}, so that a mixture is formed, which comprises organic solvent, additive, water, olefin, and hydrogen peroxide, wherein the amounts of the hydrogen peroxide and of the olefin are lower than the amounts intended to be provided in the normal run stage, and
      increasing the amount of the hydrogen peroxide and the amount of the olefin over the period of time P₂ up to the amounts intended to be provided in the normal run stage, so that a mixture 2′ is formed, and contacting the mixture 2′ over P₂ under epoxidation reaction conditions in the epoxidation zone with the heterogeneous epoxidation catalyst;
   (b′.2) removing during the respective period of time an effluent stream from the epoxidation zone, the effluent stream comprising olefin oxide, organic solvent, additive and water.
18. The start-up method of any one of embodiment 8 to 17, wherein the additive is provided in any one of (a.2.1), (b), (b′), (b.1), (b′.1), in a molar ratio M:hydrogen peroxide in the range of from 1×10⁻⁵:1 to 1×10⁻³:1, based on the molar amount of hydrogen peroxide intended to be provided in the normal run stage.
19. The start-up method of any one of embodiments 1 to 18, wherein the heterogeneous epoxidation catalyst comprises a zeolitic material having a framework structure comprising Si, O and Ti.
20. The start-up method of embodiment 19, wherein the zeolitic material comprises Ti in an amount in the range of from 0.2 to 5 weight-%, preferably in the range of from 0.5 to 4 weight-%, more preferably in the range of from 0.7 to 3 weight-%, calculated as elemental Ti and based on the total weight of the zeolitic material.
21. The start-up method of embodiment 19 or 20, wherein the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O and Ti is a titanium zeolite having an MFI framework type, an MEL framework type, an MWW framework type, an MCM-22 (S) framework type, an MCM-56 framework type, an IEZ-MWW framework type, an MCM-36 framework type, an ITQ framework type, a BEA framework type, a MOR framework type, or a mixed structure of two or more of these framework types; more preferably the zeolitic material having a framework structure comprising Si, O and Ti is a titanium zeolite having an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).
22. The start-up method of embodiment 21, wherein zeolitic material having a framework structure comprising Si, O and Ti is a titanium zeolite having an MFI framework type, which exhibits a type IV nitrogen adsorption/desorption isotherm determined at 77 K according to the method disclosed in DIN 66131 (July 1993).
23. The start-up method of any one of embodiments 19 to 22, wherein the heterogeneous epoxidation catalyst further comprises a binder, which preferably comprises silicon dioxide.
24. The start-up method of embodiment 23, wherein the heterogeneous epoxidation catalyst is in the form of a molding, preferably in the form of a strand or a granule.
25. The start-up method of embodiment 23 or 24, wherein from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.
26. The start-up method of any one of embodiments 24 to 25 wherein from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.
27. The start-up method of embodiment 26, wherein the heterogeneous epoxidation catalyst, preferably the molding, comprises the binder, calculated as SiO₂, in an amount in the range of from 1 to 95 weight-%, preferably in the range of from 3 to 70 weight-%, more preferably in the range of from 5 to 50 weight-%, more preferably in the range of from 10 to 30 weight-%, based on the total weight of the epoxidation catalyst, preferably based on the total weight of the molding and/or wherein the heterogeneous epoxidation catalyst, preferably the molding, comprises the zeolitic material in an amount in the range of from 5 to 99 weight-%, preferably in the range of from 30 to 97 weight-%, more preferably in the in the range of from 50 to 95 weight-%, more preferably in the range of from 70 to 90 weight-%, based on the total weight of the heterogeneous epoxidation catalyst, preferably based on the total weight of the molding.
28. The start-up method of any one of embodiments 1 to 27, wherein the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019).
29. The start-up method of any one of embodiments 1 to 28, wherein the hydrogen peroxide has a pH in the range of from 0 to 3.0, preferably in the range of from 0.1 to 2.5, more preferably in the range of from 0.5 to 2.3, determined with a pH sensitive glass electrode according to CEFIC PEROXYGENS H₂O₂ AM-7160 standard (2003).
30. The start-up method of embodiment 28 or 29, wherein the aqueous hydrogen peroxide solution comprises from 20 to 85 weight-%, preferably from 30 to 75 weight-%, more preferably from 40 to 70 weight-% of hydrogen peroxide, relative to the total weight of the aqueous hydrogen peroxide solution.
31. The start-up method of any one of embodiments 1 to 30, wherein the hydrogen peroxide is obtained or obtainable from an anthraquinone process.
32. The start-up method of any one of embodiments 1 to 31, wherein the organic solvent is an organic epoxidation solvent, preferably the organic solvent is selected from the group consisting of alcohol, acetonitrile, propionitrile and mixtures of two or more thereof; more preferably selected from the group consisting of alcohol, acetonitrile and mixtures of alcohol and acetonitrile; more preferably the organic solvent comprises at least an alcohol, wherein the alcohol is preferably a C₁ to C₅ mono alcohol or a mixture of two or more C₁ to C₅ alcohols, more preferably the alcohol comprises at least methanol, wherein the organic solvent more preferably comprises at least 90 weight-%, more preferably at least 95 weight-%, more preferably at least 98 weight-%, more preferably at least 99 weight-%, of methanol.
33. The start-up method of any one of embodiments 1 to 32, wherein the olefin is a C₂-C₁₀ alkene, preferably a C₂-C₅ alkene, more preferably a C₂-C₄ alkene, more preferably ethylene or propylene, more preferably propylene.
34. A process for preparing an olefin oxide comprising a normal run stage and a start-up stage, wherein the normal run stage comprises
   (A) providing olefin, hydrogen peroxide, additive water and organic solvent into an epoxidation zone comprising an heterogeneous epoxidation catalyst, so that a reaction mixture comprising olefin, hydrogen peroxide, additive, water and organic solvent is formed;
   (B) subjecting the reaction mixture from (A) to epoxidation reaction conditions in the epoxidation zone, thereby obtaining a mixture comprising olefin oxide, additive, water and organic solvent;
   (C) removing an effluent stream from the epoxidation zone, comprising olefin oxide, additive, water and organic solvent;
   wherein the start-up stage comprises
   (a) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst.
35. The process for preparing an olefin oxide comprising a normal run stage and a start-up stage of embodiment 34, wherein the additive is selected from the group consisting of potassium salt of an inorganic acid, potassium salt of an organic acid, ammonia, ammonium salt of an inorganic acid, ammonium salt of an organic acid and mixtures of two or more thereof;
   wherein the additive is preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof,
   more preferably form the group consisting of potassium dihydrogen phosphate, dipotassium hydrogen phosphate, dipotassium etidronate, ammonia and mixtures of two or more thereof;
   wherein the additive more preferably comprises at least dipotassium etidronate; wherein more preferably in the range of from 95 to 100 weight-% of the additive are dipotassium etidronate.
36. The process for preparing an olefin oxide comprising a normal run stage and a start-up stage of embodiment 34 or 35, wherein the additive is in case of ammonia provided in pure form or in aqueous solution, wherein the additive is in case of being selected from the group consisting of potassium salt of an inorganic acid, potassium salt of an organic acid, ammonium salt of an inorganic acid, ammonium salt of an organic acid or any mixture comprising two or more salts of these acids or any mixture comprising ammonia and at least one salts of these acids provided in aqueous solution;
   wherein M represents in case of a potassium salt K⁺, or, in case of ammonia or ammonium salts, M represents the sum of NH₄⁺ and NH₃.
37. The process for preparing an olefin oxide comprising a normal run stage and a start-up stage of embodiment 36, wherein the additive is selected from the group consisting of potassium salt of an inorganic acid, potassium salt of an organic acid, ammonium salt of an inorganic acid, ammonium salt of an organic acid, a mixture comprising two or more of these acid salts and a mixture comprising ammonia and at least one of these acid salts, preferably from the group consisting of potassium salt of an inorganic acid, potassium salt of an organic acid, ammonium salt of an inorganic acid, ammonium salt of an organic acid, and a mixture comprising two or more of these acid salts, and is provided as an aqueous solution, wherein the aqueous solution of additive preferably comprises in the range of from 0.01 to 5 weight-%, more preferably in the range of from 0.5 to 3 weight-%, more preferably in the range of from 1 to 2 weight-%, of additive, based on the overall weight of the aqueous solution.
38. The process for preparing an olefin oxide comprising a normal run stage and a start-up stage of embodiment 36, wherein the additive is ammonia, which is provided in pure form or in aqueous solution, wherein the aqueous solution of ammonia preferably comprises in the range of from 0.01 to 32 weight-% of ammonia, based on the overall weight of the aqueous solution.
39. The process for preparing an olefin oxide comprising a normal run stage and a start-up stage of any one of embodiments 34 to 38, wherein (a) comprises
   (a.1) providing organic solvent, an additive and water for a period of time P_{1.1} to the epoxidation zone, so that a mixture 1.1 is formed, which comprises organic solvent, additive and water and contacting the mixture 1.1 over P_{1.1} under epoxidation reaction conditions in the epoxidation zone with the heterogeneous epoxidation catalyst;
   (a.2) providing organic solvent, additive, water and olefin for a second period of time P_{1.2} to the epoxidation zone, so that a mixture 1.2 is formed, which comprises organic solvent, additive, water and olefin, and contacting the second mixture 1.2 over P_{1.2} under epoxidation reaction conditions in the epoxidation zone with the heterogeneous epoxidation catalyst.
40. The process for preparing an olefin oxide comprising a normal run stage and a start-up stage of any one of embodiments 34 to 39, wherein the additive is provided in at least one of (a), (a.1), (a.2), preferably in (a) or in (a.1), (a.2) respectively, in a molar ratio M:hydrogen peroxide intended to be provided in the normal run stage in the range of from 1×10⁻⁵:1 to 1×10⁻³:1.
41. The process for preparing an olefin oxide comprising a normal run stage and a start-up stage of any one of embodiments 34 to 40, wherein the additive is provided in at least one of (a), (a.1), (a.2), preferably in (a) or in (a.1) and (a.2) respectively, so that at the end of P₁ or P₁₂ respectively, the heterogeneous epoxidation catalyst has been contacted with a molar amount M per weight catalyst in the range of from in the range of from 2600 to 4400 μmol M/kg catalyst, preferably in the range of from 2700 to 4300 μmol M/kg catalyst, more preferably in the range of from 2800 to 4200 μmol M/kg catalyst, more preferably in the range of from 2900 to 4100 μmol M/kg catalyst, more preferably in the range of from
42. The process for preparing an olefin oxide comprising a normal run stage and a start-up stage of any one of embodiments 34 to 41, wherein the first period of time P₁ and/or the overall period of time of P_{1.1}+P_{1.2} is a period of time in the range of from 15 to 120 minutes, preferably in the range of from 45 to 100 minutes, more preferably in the range of from 55 to 90 minutes, more preferably in the range of from 60 to 85 minutes, more preferably in the range of from 75 to 80 minutes.
43. The process for preparing an olefin oxide comprising a normal run stage and a start-up stage of any one of embodiments 34 to 42, wherein the amount of olefin optionally provided in (a) and/or provided in (a.2) is lower than the amount intended to be provided in the normal run stage.
44. The process for preparing an olefin oxide comprising a normal run stage and a start-up stage of any one of embodiments 34 to 43, further comprising
   (b) after the period of time of P₁ or P_{1.2} respectively, providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst.
45. The process for preparing an olefin oxide comprising a normal run stage and a start-up stage of embodiment 44, wherein the amounts of olefin and of hydrogen peroxide provided in (b) are lower than the respective amounts intended to be provided in the normal run stage.
46. The process for preparing an olefin oxide comprising a normal run stage and a start-up stage of embodiment 44 or 45, wherein (b) comprises, preferably is,
   (b′) after the period of time P₁ or P_{1.2} respectively, providing organic solvent, additive, water, olefin and hydrogen peroxide in initial amounts to the epoxidation zone for a period of time P_{2′}, so that a mixture is formed, which comprises organic solvent, additive, water olefin, and hydrogen peroxide, wherein the amounts of the hydrogen peroxide and of the olefin are lower than the amounts intended to be provided in the normal run stage, and
      increasing the amount of the hydrogen peroxide and the amount of the olefin over the period of time P₂ up to the amounts intended to be provided in the normal run stage, so that a mixture 2′ is formed, and contacting the mixture 2′ over P₂ under epoxidation reaction conditions in the epoxidation zone with the heterogeneous epoxidation catalyst.
47. The process for preparing an olefin oxide comprising a normal run stage and a start-up stage of any one of embodiments 34 to 46, wherein the olefin is provided in (a) or in (a.2) respectively in a molar ratio relative to the molar amount of hydrogen peroxide intended to be used in the normal run stage in the range of from 0.1:1 to 4, preferably in the range of from 0.1:1 to 4:1, or in the range of rom 2.1 to 4:1.
48. The process for preparing an olefin oxide comprising a normal run stage and a start-up stage of any one of embodiments 34 to 47, wherein the olefin and the hydrogen peroxide are provided in (b) or in (b′) respectively so that the molar ratio of olefin:hydrogen peroxide in the second mixture 2 formed in (b) or in the mixture 2′ formed in (b′) respectively is in the range of from 1.1:1 to 5:1, preferably in the range of from 1.1:1 to 2:1 or in the range of from 3:1 to 5:1.
49. The process for preparing an olefin oxide comprising a normal run stage and a start-up stage of any one of embodiments 38 to 48, wherein (a.1) comprises
   (a.1.1) providing organic solvent, additive and water for a period of time P_{1.1} to the epoxidation zone, so that a mixture 1.1 is formed, which is essentially free of hydrogen peroxide, and contacting the mixture 1.1 over P_{1.1} under epoxidation reaction conditions in the epoxidation zone with the heterogeneous epoxidation catalyst;
   (a. 1.2) removing during P_{1.1} an effluent stream from the epoxidation zone, the effluent stream comprising organic solvent, additive and water;
   and/or
   wherein (a.2) comprises
   (a.2.1) providing organic solvent, additive, water and olefin for a period of time P_{1.2} to the epoxidation zone, so that a mixture 1.2 is formed, which is essentially free of hydrogen peroxide, and contacting the mixture 1.2 over P_{1.2} under epoxidation reaction conditions in the epoxidation zone with the heterogeneous epoxidation catalyst;
   (a.2.2) removing during P_{1.2} an effluent stream from the epoxidation zone, the effluent stream comprising olefin, organic solvent, additive and water.
50. The process for preparing an olefin oxide comprising a normal run stage and a start-up stage of any one of embodiments 34 to 49, wherein (b) comprises
   (b.1) after the period of time P₁ or P_{1.2} respectively providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst;
   (b.2) removing during the respective period of time an effluent stream from the epoxidation zone, the effluent stream comprising olefin oxide, organic solvent, additive and water;
   and/or
   wherein (b′) comprises
   (b′.1) after the second period of time P₁ or P_{1.2}, providing organic solvent, additive, water, olefin and hydrogen peroxide in initial amounts to the epoxidation zone for a period of time P_{2′}, so that a mixture is formed, which comprises organic solvent, additive, water, olefin, and hydrogen peroxide, wherein the amounts of the hydrogen peroxide and of the olefin are lower than the amounts intended to be provided in the normal run stage, and
      increasing the amount of the hydrogen peroxide and the amount of the olefin over the period of time P₂ up to the amounts intended to be provided in the normal run stage, so that a mixture 2′ is formed, and contacting the mixture 2′ over P₂ under epoxidation reaction conditions in the epoxidation zone with the heterogeneous epoxidation catalyst;
   (b′.2) removing during the respective period of time an effluent stream from the epoxidation zone, the effluent stream comprising olefin oxide, organic solvent, additive and water.
51. The process for preparing an olefin oxide comprising a normal run stage and a start-up stage of any one of embodiment 42 to 50, wherein the additive is provided in any one of (a.2.1), (b), (b′), (b.1), (b′.1), in a molar ratio M:hydrogen peroxide in the range of from 1×10⁻⁵:1 to 1×10⁻³:1, based on the molar amount of hydrogen peroxide intended to be provided in the normal run stage.
52. The process for preparing an olefin oxide comprising a normal run stage and a start-up stage of any one of embodiments 34 to 51, wherein the heterogeneous epoxidation catalyst comprises a zeolitic material having a framework structure comprising Si, O and Ti.
53. The process for preparing an olefin oxide comprising a normal run stage and a start-up stage of embodiment 52, wherein the zeolitic material comprises Ti in an amount in the range of from 0.2 to 5 weight-%, preferably in the range of from 0.5 to 4 weight-%, more preferably in the range of from 0.7 to 3 weight-%, calculated as elemental Ti and based on the total weight of the zeolitic material.
54. The process for preparing an olefin oxide comprising a normal run stage and a start-up stage of embodiment 52 or 53, wherein the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22 (S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O and Ti is a titanium zeolite having an MFI framework type, an MEL framework type, an MWW framework type, an MCM-22 (S) framework type, an MCM-56 framework type, an IEZ-MWW framework type, an MCM-36 framework type, an ITQ framework type, a BEA framework type, a MOR framework type, or a mixed structure of two or more of these framework types; more preferably the zeolitic material having a framework structure comprising Si, O and Ti is a titanium zeolite having an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).
55. The process for preparing an olefin oxide comprising a normal run stage and a start-up stage of embodiment 54, wherein zeolitic material having a framework structure comprising Si, O and Ti is a titanium zeolite having an MFI framework type, which exhibits a type IV nitrogen adsorption/desorption isotherm determined at 77 K according to the method disclosed in DIN 66131 (July 1993).
56. The process for preparing an olefin oxide comprising a normal run stage and a start-up stage of any one of embodiments 52 to 55, wherein the heterogeneous epoxidation catalyst further comprises a binder, which preferably comprises silicon dioxide.
57. The process for preparing an olefin oxide comprising a normal run stage and a start-up stage of embodiment 56, wherein the heterogeneous epoxidation catalyst is in the form of a molding, preferably in the form of a strand or a granule.
58. The process for preparing an olefin oxide comprising a normal run stage and a start-up stage of embodiment 56 or 57, wherein from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.
59. The process for preparing an olefin oxide comprising a normal run stage and a start-up stage of any one of embodiments 56 to 58, wherein from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.
60. The process for preparing an olefin oxide comprising a normal run stage and a start-up stage of embodiment 59, wherein the heterogeneous epoxidation catalyst, preferably the molding, comprises the binder, calculated as SiO₂, in an amount in the range of from 1 to 95 weight-%, preferably in the range of from 3 to 70 weight-%, more preferably in the range of from 5 to 50 weight-%, more preferably in the range of from 10 to 30 weight-%, based on the total weight of the epoxidation catalyst, preferably based on the total weight of the molding and/or wherein the heterogeneous epoxidation catalyst, preferably the molding, comprises the zeolitic material in an amount in the range of from 5 to 99 weight-%, preferably in the range of from 30 to 97 weight-%, more preferably in the in the range of from 50 to 95 weight-%, more preferably in the range of from 70 to 90 weight-%, based on the total weight of the heterogeneous epoxidation catalyst, preferably based on the total weight of the molding.
61. The process for preparing an olefin oxide comprising a normal run stage and a start-up stage of any one of embodiments 34 to 60, wherein the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019).
62. The process for preparing an olefin oxide comprising a normal run stage and a start-up stage of any one of embodiments 34 to 61, wherein the hydrogen peroxide has a pH in the range of from 0 to 3.0, preferably in the range of from 0.1 to 2.5, more preferably in the range of from 0.5 to 2.3, determined with a pH sensitive glass electrode according to CEFIC PEROXYGENS H2O2 AM-7160 standard (2003).
63. The process for preparing an olefin oxide comprising a normal run stage and a start-up stage of any one of embodiments 34 to 62, wherein the hydrogen peroxide is obtained or obtainable from an anthraquinone process.
64. The process for preparing an olefin oxide comprising a normal run stage and a start-up stage of any one of embodiments 34 to 63, wherein the organic solvent is an organic epoxidation solvent, preferably the organic solvent is selected from the group consisting of alcohol, acetonitrile, propionitrile and mixtures of two or more thereof; more preferably selected from the group consisting of alcohol, acetonitrile and mixtures of alcohol and acetonitrile; more preferably the organic solvent comprises at least an alcohol, wherein the alcohol is preferably a C₁ to C₅ mono alcohol or a mixture of two or more C₁ to C₅ alcohols, more preferably the alcohol comprises at least methanol, wherein the organic solvent more preferably comprises at least 90 weight-%, more preferably at least 95 weight-%, more preferably at least 98 weight-%, more preferably at least 99 weight-%, of methanol.
65. The process for preparing an olefin oxide comprising a normal run stage and a start-up stage of any one of embodiments 34 to 64, wherein the olefin is a C₂-C₁₀ alkene, preferably a C₂-C₅ alkene, more preferably a C₂-C₄ alkene, more preferably ethylene or propylene, more preferably propylene.
66. The process for preparing an olefin oxide according to any one of embodiments 34 to 65, wherein the epoxidation in (B) is carried out at an absolute pressure in the reaction zones in the range of from 0.5 to 5.0 MPa, preferably in the range of from 1.5 to 3.0 MPa, more preferably in the range of from 1.8 to 2.8 MPa.
67. The process for preparing an olefin oxide according to any one of embodiments 34 66, wherein the epoxidation in (B) is carried out at a temperature in the reaction zones in the range of from 20 to 75° C., preferably in the range of from 25 to 75° C., more preferably in the range of from 28 to 70° C., more preferably in the range of from 30 to 65° C.
68. The process for preparing an olefin oxide according to any one of embodiments 34 to 67, wherein the molar ratio of olefin:hydrogen peroxide (w/w) in the reaction mixture formed in (A) is in the range of from 1.1:1 to 5:1, preferably in the range of from 1.1:1 to 2:1 or in the range of from 3:1 to 5:1.
69. The process for preparing an olefin oxide according to any one of embodiments 34 to 68, wherein the weight ratio of organic solvent:hydrogen peroxide (w/w) in the reaction mixture formed in (A) is in the range of from 15:1 to 5:1, preferably in the range of from 12:1 to 6:1, more preferably in the range of from 12:1 to 8.5:1 or in the range of from 8:1 to 6:1.
70. The process for preparing an olefin oxide according to any one of embodiments 34 to 69, wherein the weight ratio of organic solvent:olefin (w/w) in the reaction mixture formed in (A) is in the range of from 10:1 to 1:0.1, preferably in the range of from 9:1 to 1:1, more preferably in the range of from 7:1 to 4:1 or in the range of from 1.5:1 to 1:1.
71. The process for preparing an olefin oxide according to any one of embodiments 34 to 70, wherein an additive is provided in (A), wherein the additive is selected from the group consisting of potassium salt of an inorganic acid, potassium salt of an organic acid, ammonia, ammonium salt of an inorganic acid, ammonium salt of an organic acid and mixtures of two or more thereof;
   wherein the additive is preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof,
   more preferably form the group consisting of potassium dihydrogen phosphate, dipotassium hydrogen phosphate, dipotassium etidronate, ammonia and mixtures of two or more thereof;
   wherein the additive more preferably comprises at least dipotassium etidronate; wherein more preferably in the range of from 95 to 100 weight-% of the additive are dipotassium etidronate.
72. The process for preparing an olefin oxide according to embodiment 71, wherein the additive is in case of ammonia provided in pure form or in aqueous solution, wherein the additive is in case of being selected from the group consisting of potassium salt of an inorganic acid, potassium salt of an organic acid, ammonium salt of an inorganic acid, ammonium salt of an organic acid or any mixture comprising two or more thereof or any mixture comprising ammonia and at least one thereof provided in aqueous solution;
   wherein the additive in aqueous solution releases or forms one or more cation(s) M, wherein M represents in case of a potassium salt K⁺, or, in case of ammonia or ammonium salts, M represents the sum of NH₄⁺ and NH₃.
73. The process for preparing an olefin oxide according to embodiment 71 or 72, wherein the aqueous solution of additive comprises in the range of from 0.01 to 5 weight-%, preferably in the range of from 0.5 to 3 weight-%, more preferably in the range of from 1 to 2 weight-%, of additive, based on the overall weight of the aqueous solution.
74. The process for preparing an olefin oxide according to any one of embodiments 71 to 73, wherein the additive is preferably pre-mixed with the organic solvent or with the hydrogen peroxide, more preferably pre-mixed with the organic solvent, before being provided to the epoxidation zone in (A).
75. The process for preparing an olefin oxide according to any one of embodiments 34 to 74, wherein the additive is provided in (A) in a molar ratio M:hydrogen peroxide in the range of from 1×10⁻⁵:1 to 1×10⁻³:1.
76. The process for preparing an olefin oxide according to any one of embodiments 34 to 75, wherein the epoxidation reaction conditions according to (B) comprise fixed bed conditions.
77. The process for preparing an olefin oxide according to any one of embodiments 34 to 76, wherein the epoxidation reaction conditions according to (B) comprise trickle bed conditions.
78. An olefin oxide obtained or obtainable from the process of any one of embodiments 34 to 77.

The present invention is further illustrated by the following reference examples, comparative examples, and examples.

### EXAMPLES

Chemicals

| | |
|---|---|
| Methanol | CH₃OH |
| Propylene | C₃H₅ (C₃) |
| Oxygen | O₂ |
| Nitrogen | N₂ |
| Hydrogen peroxide | H₂O₂ (HP) |
| dipotassium etidronate | K₂HEDP |
| Titanium silicalite-1 zeolite | TS-1 |

The TS-1 catalyst was prepared according to Reference Example 12 of WO 2020/074586 A1 and was used in the form of extruded strands having a diameter of 1.7 mm (cross section). The extrudates contained 75 wt.-% of TS-1 and 25 wt.-% of silicon dioxide.

### Analytic Methods

### Determination of Organic Constituents

The concentration of the constituents of the effluent (product) stream coming from the 2^{nd} separator (see Reference Example 1) was analyzed by Gas Chromatography (GC).

### Determination of Oxygen and Nitrogen Concentration

The flow of the vent stream after 2nd separator (see Reference Example 1) was measured by using a Brunkhorst flowmeter and the oxygen concentration was measured by using a Dräger Polytron 7000 detector with electrochemical sensor.

### Determination of Water Concentration

Water concentration was determined by Karl Fischer titration.

### Determination of Hydrogen Peroxide

Hydrogen peroxide was photometrically determined according to DIN38409, part 15 (June 1897).

### Determination of Total Peroxides

Total peroxides (organic ROOH and H₂O₂) were given as the active oxygen content. The active oxygen content in the samples was determined by iodometry. The following gives a general description on how the determination was performed.

Approximately 5 g of the sample, weighed to the nearest 0.1 mg, were placed in a reaction vial, flushed with argon, and 40 ml of a 1:1 acetic acid/chloroform mixture were added to dissolve the sample. The reaction vial was provided with a cooler and placed in a stirring heating block, that was already preheated to 80° C. A weak argon flow was passed through the cooler to prevent the ingress of air. After the temperature had equilibrated, 5.0 ml of a saturated potassium iodide solution (about 60.0 g potassium iodide dissolved in 100 ml deionized water) were added through the cooler and the mixture was boiled under reflux for 10 min. In the next step, 40.0 ml deionized water were added, and the sample solution was titrated with a 0.01 M thiosulfate solution while using a platinum electrode as end point indicator.

### Calculation of Hydrogen Peroxide Conversion

The H₂O₂ conversion in % was calculated according to the following equitation (I):

$${H}_{2} {O}_{2} conversion [ \% ] = {[ ( mmol hydrogen peroxide ( feed ) - mmol hydrogen peroxide ( effluent ) ) / mmol hydrogen peroxide ( feed ) ]}^{*} 100 \% \qquad ( I )$$

wherein “mmol hydrogen peroxide (feed)” means the mmol amount of H₂O₂ in the feed stream to the reactor and “mmol hydrogen peroxide (effluent)” means the mmol amount of H₂O₂ in the effluent stream coming out of the reactor.

### Reference Example 1: Experimental Reactor Setup for Example 1

127 g of TS-1 catalyst was loaded into a reaction tube of a mini-plant with a length of 180 cm and a volume of 300 ml. The tube diameter was 0.75 inch (1.905 cm), with a wall thickness of 0.07 inch (0.19 cm). In the center of the reaction tube a smaller (0.125 inch, 0.3175 cm) tube was installed, containing thermoelements for measuring the temperature over the catalyst bed. The hotspot, i.e. the zone where maximum reaction takes place, is typically located in the first half of a catalyst bed and preferably the majority of the thermoelements used is located in the first half of the catalyst bed.

Propylene was stored in 50 l gas bottles, containing dip tubes, facilitating the transfer to the mini-plant by means of 25 bar nitrogen pressure. The precise amount was measured using a Brunkhorst flow meter with a 0-500 g/h range and the flow is controlled by means of a Flowserve control-valve. Hydrogen peroxide was transferred into the reactor using a Grundfos pump DME2. The amount was determined using a balance. The measurement showed liters/minute. The respective additive solution was fed to the reactor, using a hydrogen peroxide LC pump. The precise amount was determined using a balance. For feeding the methanol a Lewa pump with a range of 0-1500 ml/h was used. Feed control was accomplished using a Lewa KMM. Nitrogen was fed using a Flowserve control-valve. The amount was measured using a Brunkhorst flow meter with a range of 0-200 NI/h. “NI/h” means norm liter per hour, wherein 1 norm liter is the amount of gas, which fills 1 liter at 0° C. and 1013 mbar (see DIN 1343 from January 1990).

Reactants, solvents, additive as indicated in detail in Example 1 and Comparative Example 1, all entered the reaction tube via a static mixer [0.25 inch (0.635 cm)-mixer], so that a combined feed stream was formed and fed to the reaction tube, wherein the feed direction was from the bottom to the top direction of the reaction tube.

The reactor effluent was passed through a 2 micrometer filter to remove fine (catalyst) particles before it was passed into the first separator. The bottom level valve controlled a level of 25% in the first separator, while the upper pressure valve set a pressure of 20 bars over the entire upstream reaction system. The second separator was also operated at a liquid level of 25%, while the upper pressure valve reduced the pressure to 2 bars. This lower pressure served for allowing the flashing of unconverted propylene, allowing a safe sample taking and having an additional safety buffer. The two separators had a volume of 2 liters each and were kept at a temperature of 5° C., using cooling water. A nitrogen stream of a gas flow of 5 NI/h was fed through the entire system (reactor->1^{st} separator->2^{nd} separator->vent-system) to maintain a sufficient gas flow in the direction of the vent to ascertain that traces of oxygen, formed by partial decomposition of H₂O₂ were flashed out and could be analyzed at the end of the vent pipe. A flowmeter was installed in the vent line, though which the gaseous vent stream coming from the 2^{nd} separator flows, to measure the flow and quantify the amount of oxygen. The liquid effluent (product stream coming from the 2nd separator) was analyzed by Gas Chromatography (GC) with respect to the compounds contained therein and their respective amounts, concentrations.

### Example 1 and Comparative Examples 1, 2, 3, 4: Start-Up Process with Feed Additive Starting Prior to Start of Propylene and Prior to Start of Feed of Aqueous Hydrogen Peroxide Solution

### Start-Up Stage

The reactor of Reference Example 1 was purged with nitrogen and pressurized at 20 bar.
1. At the beginning, methanol was fed with a flow rate of 370 g/h via the static mixer to the reaction tube, so that the reaction tube is initially flooded with methanol, wherein the feed direction was from the bottom to the top direction of the reaction tube.
2. 15 minutes later, addition of a K₂HEDP feed stream (aqueous solution of dipotassium etidronate (K2HEDP), 1.2 weight-% K₂HEDP) was started via the static mixer to the reaction tube, wherein the solution was dosed prescient to achieve a concentration of X μmol K⁺ per mol hydrogen peroxide added in step 5, i.e. the K₂HEDP feed stream was provided with a dosing rate of μmol K⁺/min as indicated in Table 1 below. The feeding was continued for a period of time (Y−15 minutes).
3. Propylene addition was started via the static mixer with a flow rate of 54 g/h, so that a combined stream comprising methanol, water, K₂HEDP and propylene was fed to the reaction tube for 15 minutes—the overall time of steps 2 and 3 being K₂HEDP feeding time Y; the values for “X” and “Y”, the flow rate and the resulting amount of potassium cations contacted with the catalyst (in kg) over the period of Y minutes are indicated below in Table 1.
4. After the 15 minutes of step 3, feeding an aqueous hydrogen peroxide solution (40 weight-% H₂O₂) with a flow rate of 65 g/h (70% of the flow rate used for normal run) to the static mixer was begun, so that a combined stream of methanol, propylene, water, hydrogen peroxide and K₂HEDP was fed to the reaction tube—from that point in time, the time on stream was counted (t_{on stream}=0 h).
5. After 24 hours, the flow rate of the aqueous hydrogen peroxide was increased to 94 g/h—step 5 initiates the beginning of the normal run stage.

TABLE 1
K₂HEDP concentration X and feed time Y

| | Comp. | | Comp. | Comp. | Comp. |
|---|---|---|---|---|---|
| | Exam- | Exam- | Exam- | Exam- | Exam- |
| Parameter | ple 1 | ple 1 | ple 2 | ple 3 | ple 4 |
| Additive dosage | 5.67 | 5.67 | 5.67 | 7.94 | 3.60 |
| rate (μmol K⁺/min) | | | | | |
| K₂HEDP_{aq} Feed | 105 | 75 | 45 | 75 | 23 h, |
| Time Y during | | | | | 45 min |
| start-up [min] | | | | | |
| amount K⁺ | 4690 | 3351 | 2010 | 4690 | 8310 |
| [μmol/kg]* | | | | | |
| X (μmol K⁺/mol | 308 | 308 | 308 | 462 | 133 |
| hydrogen per- | | | | | |
| oxide) | | | | | |

Amount K: amount of K⁺ expressed in μmol/kg catalyst, fed to the system before starting the dosage of hydrogen peroxide. The molar amount K⁺ per weight catalyst represents the molar amount of potassium cations in the K₂HEDP feed stream fed over the period of Y divided by the weight of the catalyst used (0.127 kg), expressed in μmol/kg catalyst.

### Normal Run Stage

The reaction was then carried on under normal run conditions with a combined feed stream to the reactor based on propylene fed with 54 g/h, aqueous H₂O₂ solution (40 weight-% H₂O₂) fed with a flow rate of 94 g/h, methanol fed with a flow rate of 370 g/h and additive solution 4 g/h aqueous K₂HEDP solution (1.2 weight-% K₂HEDP), wherein the flow of the additive solution was adjusted in case of Comparative Example 3 to have the ratio micromol K⁺/mol H₂O₂ of 308 μmol K⁺/mol hydrogen peroxide. The normal run was continued at least until 70 h on stream.

During start-up stage and normal run stage the water temperature setpoint was kept constant at 25° C. by using a cooling jacket circuit with water.

During start-up stage and normal run stage, starting at t_{on stream}=0 h, samples were taken from the liquid effluent (product) stream downstream of the 2^{nd} separator at specific points in time.

Selectivities for PO (propylene oxide) and MOPs (MOPs: sum of 1-methoxy-2-propanol and 2-methoxy-1-propanol) were calculated based on hydrogen peroxide. Conversion of H₂O₂ was in all cases 100% except for CE4 where no conversion was observed in the first 3 hours on stream.

The results obtained during the start-up stage are shown below in Table 2.

TABLE 2
Selectivities obtained during the start-up
stage to PO and to by-products

| | | Selectivities after | | Selectivities after | |
|---|---|---|---|---|---|
| | Amount | 6 hours on stream | | 22 hours on stream | |
| Example | of K* | PO (%) | MOPs (%) | PO (%) | MOPs (%) |
| CE1 | 4690 | 70.4 | 27.0 | 90.9 | 6.0 |
| E1 | 3351 | 83.3 | 14.8 | 95.5 | 3.8 |
| CE2 | 2010 | 72.1 | 25.2 | 90.3 | 6.4 |
| CE3 | 4690 | 61.4 | 35.5 | 94.5 | 4.8 |

| | | |
|---|---|---|
| CE4 | 8310 | No activity in the first 3 hrs. Stopped. |

Amount of K: amount of K⁺ expressed in μmol/kg catalyst, fed to the system before starting the dosage of hydrogen peroxide

### Results—Comparison of Procedure According to Example 1 Versus Comparative Examples 1, 2, 3, 4

The first data point was generated by analyzing the sample taken after 6 hours from the hydrogen peroxide start. It could be seen that the highest PO selectivity (83.3%) was reached in Example 1 when the catalyst was exposed to 308 μmol K⁺/mol HP for 75 minutes before feeding of hydrogen peroxide started. In all the Comparative Examples, the PO selectivity was significantly lower.

The second datapoint was generated by analyzing the samples taken after 22 hours and just before increasing the hydrogen peroxide load to 100%. The highest PO selectivity (95.5%) was observed again in Example 1.

Overall, it was shown that using a start-up procedure with start of additive feed prior to start of hydrogen peroxide feed helped significantly to minimize the propylene oxide losses, wherein the initial feeds comprising additive (free of hydrogen peroxide) showed the best results when conducted for a period of time in the range of from 45 to 100 minutes, more preferably in the range of from 55 to 90 minutes, more preferably in the range of from 60 to 85 minutes, more preferably in the range of from 75 to 80 minutes. Furthermore, a molar ratio of K⁺:H₂O₂ in the range of from 1×10⁻⁵:1 to 1×10⁻³:1 showed to be most favorable results regarding PO selectivity as well as regarding reduction of by-products MOPs.

Especially, it proved effective to contact the heterogeneous epoxidation catalyst prior to start of hydrogen peroxide feed with a molar amount K⁺ per weight catalyst in the range of from 2500 to 4500 μmol K⁺/kg catalyst, preferably in the range of from 2600 to 4400 μmol K⁺/kg catalyst, preferably in the range of from 2700 to 4300 μmol K⁺/kg catalyst, more preferably in the range of from 2800 to 4200 μmol K⁺/kg catalyst, more preferably in the range of from 2900 to 4100 μmol K⁺/kg catalyst, more preferably in the range of from 3000 to 4000 μmol K⁺/kg catalyst.

### CITED LITERATURE

EP 0 230 949 81
WO 2020/074586 A1

1.-13. (canceled)

14. A start-up method for a process for preparing an olefin oxide comprising a normal run stage, wherein the normal run stage comprises providing olefin, hydrogen peroxide, additive, water and organic solvent into an epoxidation zone comprising an heterogeneous epoxidation catalyst, so that a reaction mixture comprising olefin, hydrogen peroxide, additive, water and organic solvent is formed and subjecting the reaction mixture to epoxidation reaction conditions in the epoxidation zone, thereby obtaining a mixture comprising olefin oxide and organic solvent;
wherein the start-up method comprises
(a) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst;
wherein the additive releases or forms one or more cation(s) M, wherein at the end of P₁ the heterogeneous epoxidation catalyst has been contacted with a molar amount M per weight catalyst in the range of from 2500 to 4500 μmol M/kg catalyst.

15. The start-up method of claim 14, wherein the additive is selected from the group consisting of potassium salt of an inorganic acid, potassium salt of an organic acid, ammonia, ammonium salt of an inorganic acid, ammonium salt of an organic acid and mixtures of two or more thereof.

16. The start-up method of claim 14, wherein (a) comprises
(a.1) providing organic solvent, an additive and water for a period of time P_{1.1} to the epoxidation zone, so that a mixture 1.1 is formed, which comprises organic solvent, additive and water and contacting the mixture 1.1 over P_{1.1} under epoxidation reaction conditions in the epoxidation zone with the heterogeneous epoxidation catalyst;
(a.2) providing organic solvent, additive, water and olefin for a second period of time P_{1.2} to the epoxidation zone, so that a mixture 1.2 is formed, which comprises organic solvent, additive, water and olefin, and contacting the second mixture 1.2 over P_{1.2} under epoxidation reaction conditions in the epoxidation zone with the heterogeneous epoxidation catalyst.

17. The start-up method of claim 14, wherein the additive is provided in at least one of (a), (a.1), (a.2), in a molar ratio M:hydrogen peroxide intended to be provided in the normal run stage in the range of from 1×10⁻⁵:1 to 1×10⁻³:1.

18. The start-up method of claim 14, wherein the additive is provided in at least one of (a), (a.1), (a.2), so that at the end of P₁ or P_{1.2} respectively, the heterogeneous epoxidation catalyst has been contacted with a molar amount M per weight catalyst in the range of from in the range of from 2600 to 4400 μmol M/kg catalyst.

19. The start-up method of claim 14, wherein the amount of olefin optionally provided in (a) and/or provided in (a.2) is lower than the amount intended to be provided in the normal run stage.

20. The start-up method of claim 14, further comprising
(b) after the period of time of P₁ or P_{1.2} respectively, providing organic solvent, additive, water, olefin and hydrogen peroxide to the epoxidation zone for a second period of time P₂, so that a second mixture 2 is formed, which comprises organic solvent, additive, water, olefin and hydrogen peroxide, and contacting the second mixture 2 over P₂ under epoxidation reaction conditions in the epoxidation zone with the heterogeneous epoxidation catalyst.

21. The start-up method of claim 20, wherein the amounts of olefin and of hydrogen peroxide provided in (b) are lower than the respective amounts intended to be provided in the normal run stage.

22. The start-up method of claim 20, wherein (b) comprises,
(b′) after the period of time P₁ or P_{1.2} respectively, providing organic solvent, additive, water, olefin and hydrogen peroxide in initial amounts to the epoxidation zone for a period of time P_{2′}, so that a mixture is formed, which comprises organic solvent, additive, water olefin, and hydrogen peroxide, wherein the amounts of the hydrogen peroxide and of the olefin are lower than the amounts intended to be provided in the normal run stage, and
increasing the amount of the hydrogen peroxide and the amount of the olefin over the period of time P_{2′} up to the amounts intended to be provided in the normal run stage, so that a mixture 2′ is formed, and contacting the mixture 2′ over P_{2′} under epoxidation reaction conditions in the epoxidation zone with the heterogeneous epoxidation catalyst.

23. The start-up method of claim 14, wherein the olefin is provided in (a) or in (a.2) respectively in a molar ratio relative to the molar amount of hydrogen peroxide intended to be used in the normal run stage in the range of from 0.1:1 to 4.

24. The start-up method of claim 20, wherein the olefin and the hydrogen peroxide are provided in (b) or in (b′) respectively so that the molar ratio of olefin:hydrogen peroxide in the second mixture 2 formed in (b) or in the mixture 2′ formed in (b′) respectively is in the range of from 1.1:1 to 5:1.

25. A process for preparing an olefin oxide comprising a normal run stage and a start-up stage, wherein the normal run stage comprises
(A) providing olefin, hydrogen peroxide, additive water and organic solvent into an epoxidation zone comprising an heterogeneous epoxidation catalyst, so that a reaction mixture comprising olefin, hydrogen peroxide, additive, water and organic solvent is formed;
(B) subjecting the reaction mixture from (A) to epoxidation reaction conditions in the epoxidation zone, thereby obtaining a mixture comprising olefin oxide, additive, water and organic solvent;
(C) removing an effluent stream from the epoxidation zone, comprising olefin oxide, additive, water and organic solvent;
wherein the start-up stage comprises
(a) providing an organic solvent, an additive, water and optionally an olefin for a first period of time P₁ to the epoxidation zone, so that a first mixture 1 is formed, which comprises organic solvent, additive, water and optionally olefin and contacting the first mixture 1 over P₁ under epoxidation reaction conditions in the epoxidation zone with the heterogeneous epoxidation catalyst.

26. An olefin oxide obtained from the process of claim 25.
